(19) United States
(12) Patent Application Publication
(10) Pub. No.: US 2026/0232547 A1
(43) Pub. Date: Aug. 13, 2026

(54) PROTECTIVE MATERIAL FOR MITIGATING HEAT STRESS IN PLANTS VIA PASSIVE COOLING AND METHOD OF MANUFACTURE THEREOF

(71) Applicant: Lilliput Technologies Limited, London (GB)

(72) Inventors: Laura Beatriz Chaves Martínez, Heredia (CR); Andrea Ester Colina Blanco, Cartago (CR); Mauricio Herrera Rodriguez, Alajuela (CR); Yendry Corrales Ureña, Aserrí (CR); Cindy María Torres Quirós, San José (CR)

(73) Assignee: Lilliput Technologies Limited, London (GB)

(21) Appl. No.: 19/537,052

(22) Filed: Feb. 11, 2026

Related U.S. Application Data
(60) Provisional application No. 63/757,245, filed on Feb. 11, 2025.

(51) Int. Cl. A61K 8/19; C03C 17/36

(52) U.S. Cl. CPC A61K 8/19; C03C 17/366

(57) ABSTRACT

A protective material comprising a hybrid composite derived from a suspension, including an organic polymer matrix formed upon drying and an inorganic filler system embedded therein. The organic constituents may include biocompatible polymeric materials, while the inorganic filler constituents comprise a plurality of particulates including microparticles and, optionally, submicron particles and/or nanoparticles. When applied to plant surfaces and dried, the protective material forms a conformal coating configured to mitigate heat stress in plants via passive cooling, by reducing solar heat gain, and managing heat transfer between ambient air and plant tissues.

### FIELD OF THE PRESENT DISCLOURE

The present disclosure is directed to a protective and biocompatible material for mitigating heat stress in plants. More specifically, the present disclosure is directed towards a hybrid composite for mitigating heat stress in plants via passive cooling to reduce the detrimental effects arising from prolonged exposure to solar radiation.

### INTRODUCTION

Heat stress in plants has become an increasingly pressing issue, exacerbated by rising global temperatures. Currently, heat stress impacts approximately 30% of crops worldwide, with projections indicating this figure could escalate to 70% by 2045. Such an increase could result in economic losses exceeding $2,800,000,000,000 ($2.8 trillion). Since the 1960s, agricultural productivity has declined by approximately 20%, with staple crops like rice, wheat, coffee, and sorghum experiencing yield reductions of over 25% under severe heat stress. The decline in agricultural productivity, coupled with its anticipated worsening, poses a significant threat to global food security.

The underlying mechanisms contributing to heat stress in plants are multifaceted. Solar radiation, particularly in the visible (400-700 nm) and near-infrared (NIR, >700 nm) spectrums, significantly contributes to the thermal load on plant tissues. Approximately 50% of solar energy lies within the NIR spectrum, which directly heats plant surfaces. Elevated atmospheric temperatures further exacerbate thermal load by reducing the thermal gradient between plant leaves and the surrounding air, impairing the plant's ability to effectively dissipate heat. Additionally, vapor pressure deficit (VPD) plays a crucial role. Under high VPD conditions (e.g., >2.5 kPa), plants experience rapid water loss through transpiration, prompting stomatal closure to conserve moisture. Such a response diminishes transpirational cooling, inhibits photosynthesis, and intensifies thermal stress.

Recent changes in atmospheric conditions have further compounded these challenges. Over the past 15 years, reductions in atmospheric aerosols and changes in land use patterns have led to a 5-10 W/m² increase in global surface solar radiation in many regions, a phenomenon referred to as “global brightening.” This trend, which contrasts with the “global dimming” observed during the mid-20th century, has added to the complexity of managing radiation and heat stress in plants, as the rise in radiant flux further magnifies heat accumulation at the plant surface.

The problem is further exacerbated by the decreasing availability of arable land due to urbanization, soil degradation, and environmental pressures. To maintain crop yields, farmers increasingly rely on chemical inputs such as fertilizers and biocides. However, these practices often degrade soil health, causing nutrient imbalances, acidification, and water contamination. Poor soil health amplifies the effects of heat stress, creating a feedback loop that diminishes agricultural resilience.

Existing mitigation strategies include shade nets, misting systems, reflective mulches, and protective plant coatings. However, these approaches are often cost-prohibitive, limiting their applicability to high-value crops and placing additional burdens on small-scale farmers. Furthermore, existing solutions only address a portion of the problem. For instance, protective coatings typically emphasize solar reflectivity but may compromise light-transmission profiles compatible with photosynthetically active radiation (PAR) and thermal emissivity, particularly in the 8-13 µm atmospheric transmission window where plants naturally radiate heat to the sky. These approaches also do not adequately reduce convective heat transfer from hot ambient air, provide latent-heat buffering, or provide thermal capacitance to buffer diurnal temperature swings.

From a biophysical standpoint, plants already employ a rudimentary form of spectral and thermal regulation through their cuticle (a composite of waxes, lipids, and polysaccharides) that partially reflects solar radiation, scatters ultraviolet (UV) light, and emits thermal infrared energy through vibrational modes of long-chain hydrocarbons and ester groups, behaving as a grey or near-black body. However, the non-selective nature of natural cuticular emissivity and reflectance alone may not be sufficient under current climatic conditions, and stomatal closure during heat or water stress limits the plant’s ability to employ its primary cooling mechanism.

Accordingly, the present disclosure provides biocompatible strategies for improving thermal regulation in plants to effectively mitigate the harm derived from heat stress. The present disclosure also provides strategies for mitigating heat stress in plants by supporting plants to efficiently emit thermal radiation via radiative heat loss in the presence of high air temperatures. Further, the present disclosure provides strategies for mitigating heat stress in plants through controlled modulation of solar irradiance. Additionally, the present disclosure provides strategies for mitigating heat stress in plants by minimizing heat transfer between the air and plant surfaces, enabling latent-heat buffering, and providing thermal capacitance to ensure temperature differentials remain stable.

Therefore, a hybrid composite incorporating such advantageous features is provided herein as a protective material.

### SUMMARY

This summary is provided to introduce a selection of concepts in a simplified form that are further described below in the Detailed Description. This Summary is not intended to identify key features or essential features, nor is it intended to limit the scope of the claims included herewith.

The present disclosure relates, in various embodiments, to a multifunctional thermal-management system configured to form a protective, conformal, and optically tuned coating on plant surfaces for mitigating heat stress under elevated solar and atmospheric thermal loads. The disclosed system is designed to be applied by spraying and/or brush technologies or equivalent deployment modalities and, upon drying, to generate a persistent, non-occlusive coating that supports passive radiative cooling, controlled modulation of solar irradiance, reduced convective heat exchange between ambient air and leaf surface, reduced conductive heat transfer within biological tissues, and enables latent and thermal capacitance buffering. The system, in its broadest aspects, encompasses a hybrid material formulation and the resulting composite structure design for use as a protective material. The description further provides an exemplary manufacturing approach suitable for producing a stable pseudoplastic suspension for field use which, upon drying, forms the coating.

In certain embodiments, the disclosure provides a protective material comprising an inorganic phase, which may include one or more porous and non-porous ceramic filler, polar-dielectric particulates, siliceous microparticles, optionally in combination with submicron particles and nanoparticles. The inorganic constituents may be selected from titanium dioxide, zinc oxide, iron oxides, aluminum oxides, magnesium oxides, silica, calcium oxalate, mesoporous alumina, zeolitic materials, or combinations thereof, including particulates selected for their high heat capacity. In some non-limiting embodiments, the inorganic phase comprises biogenic and/or non-biogenic origin silica microparticles having diameters between 1-100 μm, optionally in combination with silica submicron and/or nanoparticulate fractions of biogenic and/or non-biogenic origin having diameters between 1-1000 nm. Such a multi-scale particle system may be engineered to provide hierarchical structures that induce size-dependent light scattering, including Mie scattering across UV, visible, and NIR wavelengths; enable tailored reflectance of solar radiation; and, in certain embodiments, support surface phonon-polariton or vibrationally enhanced mid-infrared (mid-IR) emissive modes within the 8-13 μm atmospheric transmission window. The smaller microparticles are effective for scattering visible and near-infrared solar radiation, thereby reducing radiative heat gain, while the larger microparticles provide increased emissivity within the atmospheric transparency window (8-13 μm) due to the intrinsic Si–O vibrational phonon-polariton resonance of silica near 9-10 μm. The combined dual-scale architecture enables improved passive thermal regulation by coupling broadband solar shielding with enhanced radiative cooling performance. Furthermore, owing to the significant refractive index difference between silica (n≈1.45) and the organic components of leaf tissues and cuticular waxes (typically n≈1.35-1.50), the microparticles generate strong interfacial scattering of incident solar radiation, particularly in the near-infrared region, thereby reducing radiative heat gain and limiting leaf overheating. Through these properties, the inorganic phase may contribute to reduced solar heat gain, enhanced long-wave radiative heat loss, and improved durability of the resulting coating.

In additional embodiments, the disclosure provides an organic phase comprising cellulose microfibers (CMF), microfibrillar cellulose (MFC), cellulose nanofibers, microcrystalline cellulose, carboxymethyl cellulose (CMC), or biodegradable synthetic polymers including aliphatic polyesters and water-soluble synthetic polymers, such as poly(lactic acid) (PLA), polyhydroxyalkanoates (PHA), and poly(vinyl alcohol) (PVA) optionally in combination with other polysaccharide- or biopolymer-based thickening agents, chitosan, alginate, pectin, pullulan, lanolin, xanthan gum, natural waxes, carnauba wax, stearic acid, beeswax, natural paraffins, candelilla wax, or combinations thereof. The organic phase may be hydrophilic or amphiphilic and may form, when hydrated, a colloidal suspension exhibiting pseudoplastic behavior conducive to sprayability and capable of forming hierarchical porosity upon drying, thereby contributing to light scattering. The organic phase may possess intrinsic vibrational modes (e.g., C‒O, O‒H, C‒H stretching and bending) that enhance mid-IR emissivity, while also providing structural flexibility, environmental compatibility, and light-transmission profiles compatible with photosynthetically active radiation (PAR). In some embodiments, the organic phase comprises partially hydrated or cross-linkable polymers dispersed in a treated liquid, enabling formation of a cross-linked matrix upon drying.

In various embodiments, the disclosed protective material, when formed, constitutes a biocompatible and biodegradable hybrid composite coating exhibiting a synergistic integration of the organic and inorganic phases. The protective material may display one or more of the following: high thermal infrared emissivity, optionally non-selective or selectively enhanced within the atmospheric transmission window, thereby increasing effective radiative heat dissipation and radiative conductance; low convective heat transfer at the leaf-air interface; latent-heat buffering associated with water content and optionally phase-change domains; increased effective heat capacity for moderating diurnal temperature swings; controlled optical properties permitting PAR transmission; and selective attenuation, scattering, or reflection of UV, visible, and NIR radiation. The protective material may further promote stable adhesion to hydrophobic or structured plant cuticles through hydrogen bonding, van der Waals interactions, or surface-active adhesion promoters, while preserving gas-exchange pathways required for physiological function. In certain embodiments, the dried composite degrades into non-toxic constituents and may be compatible with soil microbiota.

In further embodiments, the disclosure incorporates an exemplary manufacturing process capable of producing stable suspensions with rheological properties suitable for spraying, brushing, dipping, casting or combinations thereof or other forms of deposition. Such process may include controlled dispersion of organic polymers and inorganic fillers into a treated liquid, regulation of polyanionic characteristics of cellulose derivatives such as CMF and MFC grades or CMC, modulation of pseudoplastic flow behavior, and maintenance of stability to ensure uniform particle distribution and reproducible surface interactions. Additional steps may include acid-salt pore-forming reactions, cross-linking, or post-dilution procedures to achieve desired viscosities, particle distributions, and coating performance.

Collectively, the materials, formulations, and process described in this disclosure provide a versatile, environmentally compatible, and functionally engineered solution for mitigating plant heat stress through passive radiative cooling, selective solar-radiation management, and improved plant surface thermal management.

### BRIEF DESCRIPTION OF THE DRAWINGS

The incorporated drawings, which are incorporated in and constitute a part of this specification exemplify the aspects of the present disclosure and, together with the description, explain and illustrate principles of this disclosure.

FIG. 1 illustrates an exemplary method for manufacturing a hybrid composite protective material configured to mitigate heat stress in plants.

FIG. 2 shows the apparent viscosity as a function of shear rate for example suspensions comprising an organic polymer base dispersion with an optional additive and different inorganic particulate fillers.

FIGS. 3A-3B illustrate scanning electron microscopy (SEM) images of representative dried coating microstructures. FIG. 3A shows a porous, interconnected fibrillar network with dispersed particulate aggregates. FIG. 3B shows bead-like particulate domains embedded within a fibrillar matrix.

FIGS. 4A-4B show optical characterization data of a coating in the visible to near-infrared region. FIG. 4A shows total (TT) and diffuse transmittance (DT), as well as total reflectance (TR) and diffuse reflectance (DR) of a coating. FIG. 4B shows comparative total reflectance of partially coated versus uncoated regions of a leaf.

FIGS. 5A-5B show mid-infrared spectral emissivity data in the atmospheric window (approximately 8-13 µm). FIG. 5A shows emissivity of a dried coating. FIG. 5B shows comparative emissivity of an uncoated leaf and the same lead after application of the coating.

FIG. 6 shows a time-series plot of leaf surface temperature under open-sky outdoor exposure, comparing a coated region and an uncoated region on the same lead together with local air temperature.

FIG. 7 shows a time-series plot for an uncoated plant and a coated plant during exposure in a ventilated pseudo-stagnation enclosure.

FIGS. 8A-8B depict infrared thermography (FIG. 8A) and a corresponding photograph (FIG. 8B) of a plant under outdoor irradiance (862 W·m⁻²), ambient air temperature 33 °C, relative humidity 66%, and equal soil humidity (85%). FIG. 8A shows infrared thermography of an uncoated plant (control) and a coated plant (treated). IR images captured at 1m distance and emissivity setting ε = 0.95. FIG. 8B shows the corresponding photograph of an uncoated plant (control) and a coated plant (treated).

FIG. 9 illustrates comparative plant growth outcomes for treated plants (right) relative to untreated plants (left) after application of a protective material.

### DETAILED DESCRIPTION

In the following detailed description, reference will be made to the accompanying drawing(s), in which identical functional elements are designated with like numerals. The aforementioned accompanying drawings show by way of illustration, and not by way of limitation, specific aspects, and implementations consistent with principles of this disclosure. These implementations are described in sufficient detail to enable those skilled in the art to practice the disclosure and it is to be understood that other implementations may be utilized and that structural changes and/or substitutions of various elements may be made without departing from the scope and spirit of this disclosure. The following detailed description is, therefore, not to be construed in a limited sense.

It is noted that description herein is not intended as an extensive overview, and as such, concepts may be simplified in the interests of clarity and brevity.

All documents mentioned in this application are hereby incorporated by reference in their entirety. Any process described in this application may be performed in any order and may omit any of the steps in the process. Processes may also be combined with other processes or steps of other processes.

### Definitions

The phrase “and/or”, as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer. According to some embodiments, to A without B (optionally including elements other than B); According to some embodiments, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of” “Consisting essentially of,” when used in the claims, shall have its ordinary meaning as used in the field of patent law.

As used herein, the phrase “integer from X to Y” means any integer that includes the endpoints. That is, where a range is disclosed, each integer in the range including the endpoints is disclosed. For example, the phrase “integer from X to Y” discloses 1, 2, 3, 4, or 5 as well as the range 1 to 5.

As used herein, the term “acidification” refers to a process where the soil pH drops (i.e., becomes more acidic, below pH 7.0) due to nutrient leaching, acid rain, fertilizers, etc. Acidification lowers soil fertility, enhances the mobility of heavy metals, reduces nutrient availability, and stunts root growth, causing poor plant health.

As used herein, the term “atmospheric window” or “atmospheric transparency window” refers to one or more specific, relatively narrow ranges of electromagnetic radiation wavelengths, including but not limited to the thermal infrared region (about 8-13 µm), that allow thermal radiation to pass through Earth’s atmosphere without being absorbed by greenhouse gases.

As used herein, the term “base dispersion” refers to a uniform distribution of reinforcements, such as organic or inorganic fibers or nanofillers, within a matrix material.

As used herein, the term “biocides” refers to chemical substances or microorganisms used to kill or control pests, pathogens, and unwanted vegetation. They act as protective agents against bacteria, fungi, and insects to preserve crop health.

As used herein, the term “biocompatible” refers to materials that are well tolerated by living systems.

As used herein, the term “biodegradable” refers to materials that naturally decompose into simple elements.

As used herein, the term “biogenic” refers to substances, materials, or compounds that are directly produced by, derived from, or synthesized through the natural biological processes of living organisms, including inorganic or mineral phases bioaccumulated, biosynthesized, or physiologically deposited within biological tissues, and retaining their inherent biogenic structural characteristics following extraction or processing

As used herein, the term “cell turgidity” refers to the firm, swollen state of plant cells caused by high internal water pressure, known as turgor pressure, pushing the cell membrane against the rigid cell wall. Cell turgidity is essential for plant structural integrity, enabling non-woody plants to stand upright and maintain rigidity. Turgidity results from water entering the central vacuole via osmosis.

As used herein, the term “colloid suspension” refers to a heterogenous mixture where insoluble particles are dispersed throughout another substance, remaining suspended without settling over time. Unlike a suspension, where particles are large enough to settle out over time, particles in a colloid range from 1-1000 nm and remain kinetically stable over time. Examples of colloid suspensions include, but are not limited to, hydrogels, emulsions, aerosols, foam, and sol.

The term "compatible" as used herein refers to components of a composition that are capable of being combined with each other in a manner such that there is no interaction that would substantially reduce the efficacy of the composition under ordinary use conditions.

The term “component” as used herein, is meant to refer to a constituent part, element or ingredient.

The term “composition” as used herein, is meant to refer to a material formed by a mixture of two or more substances.

As used herein, the term “ hybrid composite” refers to a material comprising distinct organic and inorganic phases that remain as separate domains within the composite structure designed to be applied to plants, comprising organic and inorganic materials designed to provide sustainable, high-performing, thermally protective, and/or insulative properties for enhanced thermal stability and protection against extreme heat that would otherwise damage plants.

As used herein, the term “cross-link” and its various grammatical forms refers to the process of chemically joining two or more molecules, typically polymer chains, by covalent or ionic bonds to form a network. This method increases molecular weight, and creates stronger, more rigid, more durable substances/materials.

As used herein, the term “cuticle” refers to the protective, waxy, outermost layer covering the aerial parts of land plants (e.g., leaves, stems, flowers, fruits) that acts as a barrier against water loss and environmental stresses like UV radiation, pathogens, and extreme temperatures, allowing plants to survive on land by controlling water and gas exchange. The natural cuticle is composed mainly of cutin (i.e., a polymer of fatty acids) and waxes.

As used herein, the term “deionized water” or “DI water” refers to water that is highly purified and has nearly all of its dissolved mineral ions (e.g., sodium, calcium, iron, chloride) removed through an ion exchange process.

The term “derived from” as used herein encompasses any method for receiving, obtaining, or modifying something from a source of origin.

As used herein, the term “fertilizer” refers to any material of natural or synthetic origin applied to soil or plant tissues to supply essential nutrients for growth. These substances enhance soil fertility and provide crucial nutrients (primarily nitrogen, phosphorus, and potassium) that are critical for plant health, development, and productivity.

As used herein, the term “filler” refers to solid, micro- or nano-scale particulate material added to a matrix to improve the overall performance of a composite.

As used herein, the term “global brightening” refers to the increased amount of solar radiation (i.e., sunlight) reaching the Earth’s surface.

As used herein, the term “global dimming” refers to a decrease in solar radiation (i.e., sunlight) reaching the Earth’s surface.

As used herein, the term “heat capacity” refers to the amount of heat energy needed to raise the temperature of a substance by one degree. Heat capacity is a measure of how much thermal energy can be stored, with a higher heat capacity meaning more energy is required for the same temperature change.

As used herein, the term “heat stress” refers to damage caused by sustained high air or soil temperatures exceeding a species’ optimal growth range, which disrupts physiological and biochemical processes. Heat stress may result in impaired photosynthesis, slowed growth, premature flowering, decreased pollen production, and severe symptoms such as wilting, leaf scorching, and fruit sunscald.

As used herein, the term “inorganic” refers to compounds that do not contain primarily carbon. These substances typically include minerals, metals, salts, and oxides and are generally held together by ionic bonds. The term “inorganic material” or “inorganic matter” refers to substances that do not contain carbon-hydrogen bonds and are typically derived from non-living sources, such as minerals, rocks, or metal ores. They generally exclude the complex, carbon-based molecules found in living organisms.

As used herein, the term “latent-heat buffering” refers to the process where a substance absorbs or released large amounts of heat during a phase change (e.g., melting or freezing) without changing its own temperature.

As used herein, the term “organic” refers to compounds containing carbon atoms, typically bonded to hydrogen, oxygen, nitrogen, or sulfur.

As used herein, the term “plant coating” or “protective plant coating” refers to an external, often spray-on layer that are applied to plants to shield plants from environmental stresses like drought, frost, winter burn, and UV radiation. These coatings typically reduce water loss, prevent damage from bacteria and pests, and improve overall plant survival.

As used herein, the term “phonon polariton” refers to a type of quasiparticle that can form in polar dielectric materials due to coupling of transverse optical phonons modes and photons. Phonon polaritons occur at frequencies where the electromagnetic field interacts resonantly with lattice vibrations, typically within the mid-infrared spectral region.

As used herein, the term “phonon polariton-enhanced thermal emission” refers to the increase in thermal radiation from polar dielectric materials mediated by coupled photon-phonon quasiparticles.

As used herein, the term “photosynthetically active radiation” or “PAR” refers to the specific range of sunlight (around 400-700 nm) that plants, algae, and cyanobacteria use to perform photosynthesis.

As used herein, the term “polymer” refers to a large molecule (macromolecule) composed of long chains or networks of smaller, repeating chemical units called monomers.

As used herein, the term “polysaccharide” refers to complex carbohydrates composed of long chains of monosaccharide units (i.e., simple sugars) linked together by glycosidic bonds.

The term “preserve” and its other grammatical forms as used herein refers to maintaining or keeping in an unaltered condition.

As used herein, the term “processed from” refers to a material, matter, ingredient, component, etc. that has undergone specific intentional treatments, e.g., cooking, refining, milling, preserving, etc. to change its original state.

As used herein, the term “protective plant coatings” refers to both natural waxy layers and applied, engineered, or organic films on leaves, stems, and fruits that protect against environmental stresses, pests, diseases, and water loss.

As used herein, the term “radiative cooling” or “zero-energy cooling” or “passive cooling” refers to a natural, passive process where surfaces release heat allowing materials to cool below the ambient air temperature without energy input.

As used herein, the term “reflective mulch” refers to shiny, light-colored, metallic or otherwise reflective materials placed on the soil around plants to reflect sunlight, specifically UV light, up into the plant canopy. Reflective mulches are used to confuse pests, enhance photosynthesis, and manage soil temperature.

As used herein, the term “resilience” refers to the capacity to withstand, recover from, and adapt to environmental, climatic, or anthropogenic stressors, e.g., droughts, pests, fires, nutrient deficiencies, etc., while maintaining essential growth and function.

As used herein, the term “shade nest” refers to a specialized agricultural, horticultural, and gardening material designed to protect plants from excessive heat, sunlight, and harsh environmental conditions.

As used herein, the term “solar radiation” refers to the electromagnetic energy emitted by the sun, specifically visible light, UV, and infrared, that drives vital physiological processes. Solar radiation serves as the primary energy source for photosynthesis, regulating plant growth, development, heat balance, and metabolism.

As used herein, the term “stomata” or “stoma” refers to microscopic pores located on the epidermis of leaves and stems that facilitate gas exchange and regulate transpiration. Surrounded by special guard cells, these structures open and take in carbon dioxide for photosynthesis and close to limit water loss, acting as crucial regulators for plant survival. Stomata are comprised of a pore bordered by two guard cells that change shape to open and close the aperture. Guard cells open and close the stomata in response to environmental and physiological cues.

As used herein, the term “thermal conductivity” refers to a material’s ability to transfer or conduct heat.

As used herein, the term “thermal emissivity” refers to the efficiency of a plant’s surface at emitting stored thermal energy.

As used herein, the term “thermal load” refers to the total heat energy (e.g., from solar radiation, air temperature, and environmental sources) absorbed by a plant, influencing its physiological processes, growth rate, and development. Thermal load determines heat stress levels, requiring plants to balance energy for photosynthesis against potential damage from excessive heat. Plants require a specific accumulation of heat units (measured in degree-days or hours) to progress through development stages, such as germination, flowering, and ripening. High thermal loads can cause leaf temperature to exceed air temperatures, potentially causing heat stress and reduced growth.

As used herein, the term “transpiration” refers to the process of water movement from the roots, through a plant’s vascular system, and its final evaporation as water vapor into the atmosphere, primarily through stomata. Transpiration is a passive, vital process that facilitates nutrient transport, maintains cell turgidity, and cools the plant. The mechanism of transpiration comprises water being absorbed by the roots from the soil, traveling up the xylem, and exiting as vapor, creating a suction force (i.e., tension) that pulls more water from the roots. Most transpiration occurs through stomata, but can also occur via the cuticle (i.e., leaf surface) and lenticels (i.e., pores in the stem bark).

As used herein, the term “vapor pressure deficit” or “VPD” refers to the difference between the moisture currently in the air and the maximum moisture the air can hold when saturated at a specific temperature. VPD represents the drying power of the air and is crucial for measuring plant transpiration rates, nutrient uptake, and stomatal behavior. In regard to transpiration, VPD measures the vapor pressure difference between the moist interior of a leaf and the surrounding air. Higher VPD indicates drier air, prompting high transpiration rates (i.e., water loss). Optimal VPD is about 0.4-1.6 kPa and balances water loss with nutrient uptake and carbon dioxide absorption. High VPD is above 1.5 kPa and causes excessive transpiration, causing plant stress, potential wilting, and stomatal closure to conserve water, which reduces photosynthesis. Low VPD is below 0.5 kPa and occurs in high humidity, reducing transpiration, which can slow growth, limit nutrient movement, and cause diseases or guttation (i.e., water that is pushed out of leaf edges).

### Embodiments

Aspects of the present disclosure may relate to a hybrid composite referred to herein as a protective material for heat stress mitigation in plants. In particular, the protective material may aid in mitigating heat stress in plants that are exposed to solar radiation.

In an embodiment, the protective material may be applied to foliage and/or other above-ground plant tissues using spraying techniques (e.g., ground sprayers, aerial application, crop dusters, or other suitable equipment). In certain embodiments, the protective material is applied predominantly to the adaxial (upper) leaf surface, for example to reduce incident solar loading while reducing the likelihood of obstructing stomata located on the abaxial (lower) surface. Upon coating said plant surfaces, such protective material may create a localized cooling effect around the plant surface facilitating improved temperature regulation.

In certain embodiments, the protective material may be applied in partial, discontinuous, or patterned coverage (e.g., stripes, dots, reticulated patterns, or otherwise non-continuous coating) to provide cooling benefits while maintaining gas exchange and transpiration. In some embodiments, coverage may be selected based on crop type, leaf morphology, and environmental conditions.

In some embodiments, the protective material may leverage zero-energy cooling to improve plants’ resilience to changing atmospheric conditions. For example, the protective material may improve said plant’s radiative heat dissipation by reducing solar heat absorption through UV and NIR radiation, and by enhancing effective radiative conductance, thereby enabling heat dissipation via thermal emission in the mid-IR atmospheric transmission window (8-13 µm). However, despite reflecting UV and/or NIR radiation, the composite may enable PAR to transmit through said composite and reach the surface of a plant.

In some embodiments, the protective material may provide thermal management through at least one of: (i) high thermal infrared emissivity, optionally non-selective or selectively enhanced within the atmospheric transmission window, thereby increasing effective radiative heat dissipation and radiative conductance (ii) low convection which may minimize heat transfer from hot ambient air to the plant surface and slow temperature rise during peak heat conditions; (iii) latent-heat buffering, which may be provided by water content and may optionally include phase-change domains that absorb and release heat near a designed transition temperature to reduce temperature peaks; (iv) increased effective heat capacity, which may add greater resistance to temperature change so that the plant-coating system warms more slowly and experiences smaller diurnal temperature swings; and (v) optical modulation of incident radiation, including selective reflection, scattering, absorption, and/or transmission of solar radiation to alter the plant surface energy balance.

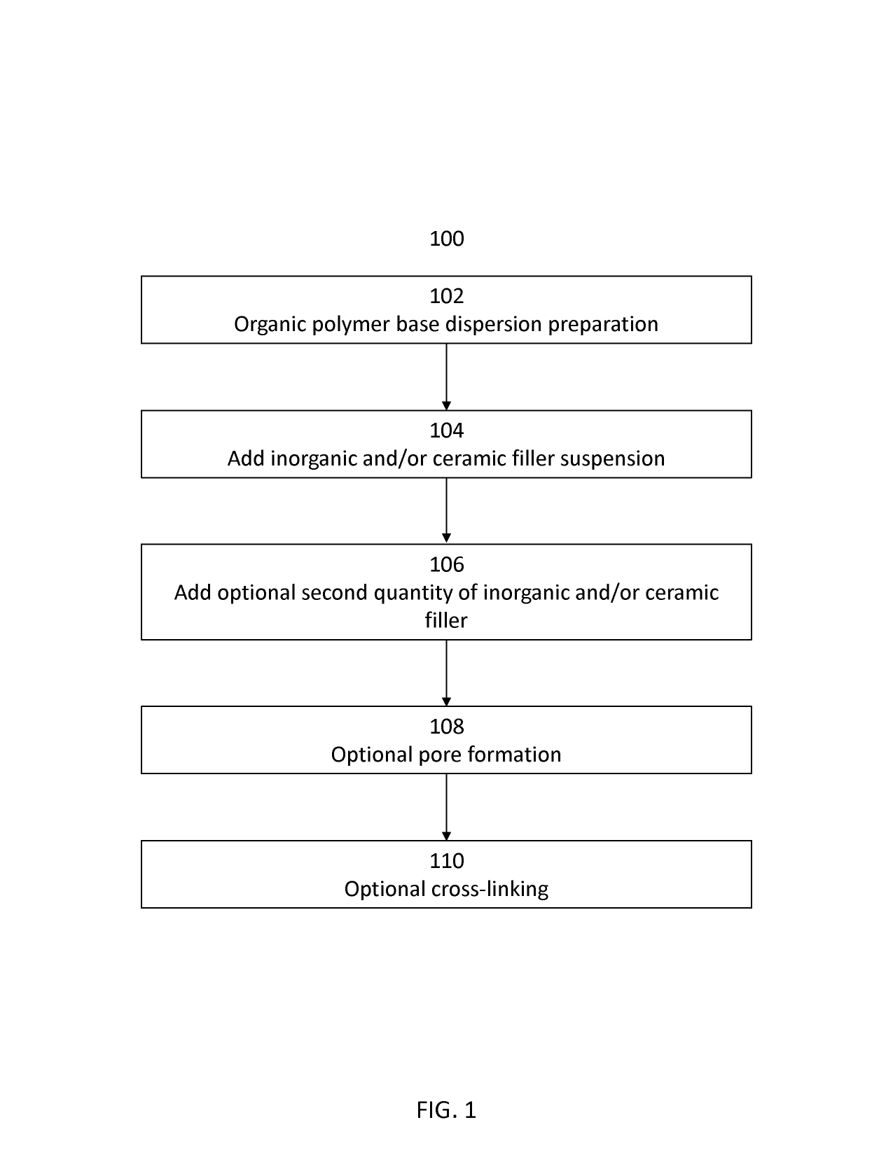

Referring to FIG. 1, another exemplary method for manufacturing a hybrid composite referred to herein as a protective material for mitigating heat stress in plants 100 may be illustrated.

In certain embodiments, the manufacturing comprises defining and controlling specifically engineered operating conditions to induce a pseudoplastic (shear-thinning) rheological behavior in a suitable suspension, wherein such operating conditions include, without limitation, predetermined ranges of shear rate, deformation gradient, mixing energy, and hydrodynamic regime, configured to facilitate application, dispersion, and stability of the system. Within this framework, the operating conditions and chemical composition are co-designed such that the shear-thinning behavior enables efficient processing and application of the material without materially compromising high infrared emissivity, selective solar radiation management, or the overall thermal performance of the coating in its applied and dried states.

The method 100 may be comprised of four stages: (1) preparation of an organic polymer base dispersion; (2) introduction of inorganic and/or ceramic fillers to the organic polymer base dispersion, thereby forming a suspension (3) optional pore formation and structural development within the suspension; (4) optional cross-linking of the suspension to enhance stability, thereby forming a crosslinked protective material.

The method 100 may be comprised of at least a first step 102. In the first step 102, the organic polymer base dispersion may be prepared. To illustrate, a quantity of organic polymers (e.g., cellulose nano or microfibers, chitosan, alginate, pectin, pullulan, xylan, etc.) may be mixed with a first quantity of a treated liquid (e.g., water). The combination of organic polymers and the treated liquid may be mixed together in a mixing reactor equipped with a mechanical stirrer. As a nonlimiting example, 0.1-2.5 w/w%, inclusive, of CMC with degree of substitution 0.7 - 1.5 may be added to the treated liquid (e.g., deionized water) and stirred until homogenous in a mixing reactor. Particularly, 0.1-2.5g, inclusive, of organic polymers , and 95-100g, inclusive, of the treated liquid (5-1000 µS/cm) may be mixed.

Nonlimiting examples of a treated liquid include, tap water, deionized water, distilled water, filtered water, softened water, purified water, aerated water, etc.

In an alternative embodiment, the organic polymer base dispersion may be created by mixing the quantity of organic polymers optionally obtained, derived, or processed from natural extracts, with a quantity of polysaccharides, and/or a quantity of biodegradable polymers and/or quantity of natural waxes. Thus, as a nonlimiting example, the organic polymer base dispersion may be created from a combination of two or more of, 0.25-2g, inclusive, of nanocrystalline cellulose (CNC), 0.5-2.5g, inclusive, of cellulose nanofibers (CNFs), 0.5-3g, inclusive, of microcrystalline cellulose (MCC), 0.3-2g, inclusive, of CMC, 0.3-3g, inclusive, of (CMF/MFC), 0.3-2g, inclusive, of sodium alginate, 0.3-3g, inclusive, of chitosan, 0.05-1g, inclusive, of xanthan gum, 0.05-5g, inclusive, of poly(lactic acid) (PLA), 0.01-3g, inclusive, of polyhydroxyalkanoates (PHA), 0.03-3g, inclusive, of poly(vinyl alcohol) (PVA), 0.01-5g, inclusive, of a natural wax, and/or 95-100g, inclusive, of the treated liquid.

In embodiments where the organic polymer base dispersion is comprised of CNC, CNF, MCC, CMC, CMF/MFC or any combination thereof, the organic polymer matrix, obtained upon drying the organic polymer base dispersion, may exhibit radiative cooling properties. That is, the organic polymer matrix may aid in reflecting solar radiation (e.g., UV and IR light) and/or dissipating heat as thermal radiation through an atmospheric window.

The radiative cooling (alternatively, “zero-energy cooling”) properties may be a product of the organic polymer matrix’s ability to emit thermal infrared radiation. For example, CMF/MFC can present semicrystalline domains and hydroxyl-rich backbones that provide vibrational modes in the mid-IR; these features can increase hemispherical emissivity within the atmospheric transmission window, facilitating thermal radiation to the sky. In addition, the fibrous network can form a porous morphology upon drying, which increases internal surface area and multiple scattering of thermal radiation, thereby promoting effective mid-IR emission. Collectively, the intrinsic vibrational bands and the porous, fibrous structure of the CMF/MFC matrix may support passive radiative heat loss from coated plant surfaces.

Such an organic polymer matrix may offer improvements over traditional plant coatings by allowing the plant to maintain a stable near-surface thermal environment. As a nonlimiting example, the light reflecting properties of the organic polymer matrix, in conjunction with the zero-energy cooling, may reduce solar heat gain and dissipate stored heat. Furthermore, the fibrous and porous structure may reduce conductive heat transfer within biological tissues..

The method 100 may be further comprised of a second step 104, wherein a first quantity of inorganic fillers optionally obtained, derived, or processed from natural extracts, are mixed into the organic polymer base dispersion, thus forming a suspension. The order of step 102 and 104 may be inverted in certain embodiments, depending on the type of organic polymer selected, in order to improve the rheological properties of the resulting suspension.

In an embodiment, the first quantity of the inorganic and/or ceramic fillers may be comprised of at least one of a plurality of microparticles of biogenic and/or non-biogenic origin having a diameter between 1-100 µm, inclusive.

In another embodiment, the inorganic and/or ceramic fillers may be comprised of an inorganic and/or ceramic constituents or combinations thereof, including particulates selected for their high heat capacity. In certain embodiments, the inorganic fillers may further include polar-dielectric particulates capable of supporting surface phonon–polariton modes. Specifically, the inorganic and/or ceramic fillers may include at least one of titanium dioxide (TiO₂), silica (SiO₂), calcium oxalate (CaC₂O₄), zinc oxide (ZnO), iron oxide (Fe₂O₃), aluminum oxide (Al₂O₃), and magnesium oxide (MgO).

In a nonlimiting example, 0.05-10 w/w%, inclusive, of the plurality of microparticles may gradually be added to the mixing reactor containing the organic polymer base dispersion. Specifically, 0.05-10g, inclusive, of silica microparticles may be added.

The combination of the first quantity of inorganic and/or ceramic fillers with the organic polymer base dispersion may be mixed at a first power level for a first period of time. For instance, the inorganic and/or ceramic fillers, and the organic polymer base dispersion may be mixed in the mixing reactor at 20-130W for 10-40 minutes, inclusive. Alternatively, the inorganic and/or ceramic fillers and the organic polymer base dispersion may be mixed in the mixing reactor at any power level for as long as it takes for said mixture at shear rates from 10⁻⁴-10⁴ s⁻¹ to reach a viscosity of 40-10⁶ mPa·s, inclusive, and/or until homogeneity is reached.

Upon reaching the said viscosity between 40-10⁶ mPa·s, inclusive, or homogeneity, the l suspension has been formed.

The method 100 may be comprised of an optional third step 106. In the third step 106, a second quantity of inorganic and/or ceramic fillers may optionally be mixed into the suspension.

In an embodiment, the second quantity of the inorganic fillers may be comprised of a plurality of submicron particles and/or a plurality of nanoparticles having a diameter between 1-1000 nm.

In a nonlimiting example, 0.01-0.5 w/w%, inclusive, of the plurality of submicron particles and/or the plurality of nanoparticles may optionally be added in this step to the suspension. In particular, 0.01-0.5g, inclusive, of silica particles having a diameter between 1-1000 nm, inclusive, may be added to the suspension.

Subsequent to the addition of the second quantity of inorganic and/or ceramic fillers, the suspension may be mixed at a second power level for a second period of time. As a nonlimiting example, the suspension may be mixed at 20-130W for 5-30 minutes, inclusive.

After mixing the suspension of step 106, said suspension may have a viscosity between 40-10⁶ mPa·s, inclusive, or may be mixed until homogeneity is reached

In an embodiment, both the first and second quantities of inorganic and/or ceramic fillers may enhance radiative cooling properties and/or control solar radiation of the protective material. To illustrate, the combination of the plurality of the microparticles, optionally in combination with submicron particles and/or nanoparticles may create a hierarchal particle system having a multi-scale, light reflective structure when embedded within the organic polymer matrix, which is obtained upon drying the suspension.

In some embodiments, in said hierarchal particle system, the first and second quantities of inorganic and/or ceramic fillers may work in tandem to scatter and/or reflect light of various wavelengths. In some embodiments, the plurality of submicron particles and the plurality of nanoparticles may scatter and/or reflect UV and visible light, while the plurality of microparticles may scatter and/or reflect IR light and may dissipate heat through the atmospheric window. In some embodiments, the plurality of the microparticles, submicron particles, and nanoparticles work together to maximize solar reflectivity across the UV and/or IR ranges and emissivity within the mid-IR.

In some embodiments, despite reflecting UV and IR light, the protective material may simultaneously allow sufficient PAR to reach the plant surface. In some embodiments, the protective material may maintain transmitted PAR within a range compatible with photosynthetic activity, including values approaching the light saturation point characteristic of many crop species, wherein the transmitted PAR is predominantly diffuse rather than specular.

The method 100 may be comprised of an optional fourth step 108, wherein an acid, a protein and/or a salt may be mixed into the suspension, thus inducing pore formation and structural development within said suspension.

For example, the acid may include organic acids, such as, acetic acid (CH₃CO₂H) and citric acid (C₆H₈O₇), as well as mineral acids including hydrochloric acid (HCl), etc. The protein may include polypeptide materials, such as albumins, casein, or plant-derived proteins. Additionally, the salt may include ammonium bicarbonate ((NH₄)HCO₃), sodium bicarbonate (NaHCO₃), etc.

In one embodiment, 0.8-1.2 w/w%, inclusive, of 1M HCl may be added to the suspension under shear rate 10⁻²-10³ s⁻¹at 25-55°C, inclusive, during 10 min. Shortly thereafter, 0.9-1.1 w/w%, inclusive, of NaHCO₃ may also be added in this step. However, those having ordinary skill in the art will appreciate that any suitable acid and/or salt alternative may be utilized.

In a further embodiment, when added to the suspension, the acid and the salt may spontaneously react. During said reaction, carbon dioxide (CO₂) may be produced. Specifically, in an embodiment where 0.8-1.2g, inclusive, HCl and 0.9-1.1g, inclusive, NaHCO₃ were added to the suspension, 0.05-1.5g, inclusive of CO₂ may be released from the suspension.

The foregoing CO₂ release values are illustrative and may vary depending on the total acid equivalents present in the formulation, including acid functionalities of polymeric components (e.g., carboxyl groups in acid-form carboxymethyl cellulose) and/or residual acidity from processing. Consequently, upon release of said CO₂ the suspension may form a porous structure comprised of a plurality of micro and/or nanopores. Accordingly, the amount and rate of CO₂ evolution, and the resulting porosity, may depend on the identity and concentration of the acid source(s), the carbonate/bicarbonate salt selected, and mixing conditions. Once the suspension has reached a viscosity of 300-10⁴ mPa·s, inclusive, the structural development of the suspension is complete.

The porous structure of the protective material, obtained upon drying the suspension following pore formation, when applied to the surface of a plant, may facilitate improved thermal dissipation upon said surface. In addition to improving thermal dissipation, the porous structure of the protective material may transiently retain thin films of moisture on plant surfaces, enabling controlled evaporative cooling and providing a latent-heat buffering effect.

To illustrate, the porous structure may increase both the surface area and capillary pathways of the protective material as a whole, which in turn, allows the protective material to transiently retain thin films of moisture on plant surfaces relative to untreated tissue.

Additionally, in general, hydrophilic and/or amphiphilic components (e.g., cellulose-based fibers, waxes, etc.), such as those comprising the protective material, are able to absorb and retain water within their molecular structure via hydrogen bonding. Thus, the combination of the porous structure and the hydrophilicity of the protective material components may enable the protective material to temporarily hold and gradually release water and thus may improve evaporative cooling on said plant’s surface.

In a further embodiment, the protective material porous structure may allow water vapor to escape from the plant’s surface, through said structure, to the atmosphere via transpiration. Thus, the porous structure ensures that the protective material does not hinder plant transpiration when coated on said plant’s surfaces.

Moreover, the porous structure of the protective material may allow gas exchange between the plant surface and the atmosphere. Specifically, CO₂ and Oxygen (O₂) may be freely exchanged between the plant surface and the atmosphere and thus ensures that the protective material does not hinder plant photosynthesis.

The method 100 may be comprised of an optional fifth step 110. In said fifth step 110, a cross-linking agent may be mixed into the suspension to stabilize it, and thereby forming a crosslinked protective material. To illustrate, the suspension may be added to a heating tank under shear rate 10⁻²-10³ s⁻¹ and heated at 25-50 °C, inclusive, for 10 minutes, and the cross-linking agent may subsequently be added to the same tank. Specifically, 0.18-0.22 w/w%, inclusive, or 0.18-0.22g, inclusive, of tannic acid (i.e., the cross-linking agent) may be added to the suspension.

The cross-linking agent may increase the stability of the suspension. For instance, natural cross-linking agents, such as, but not limited to, citric acid, trisodium 1,3,5,2,4,6-trioxatriphosphinane-2,4,6-triolate 2,4,6-trioxide, calcium dichloride, methyl (1R,4aS,7aS)-1-hydroxy-7-(hydroxymethyl)-1H,4aH,5H,7aH-cyclopenta[c]pyran-4-carboxylate, and tannic acid, may be incorporated into the suspension to promote improved intermolecular bonding of the organic polymers added in step 102. Consequently, the protective material may display improved resistance to degradation from external elements including precipitation and UV exposure.

After the cross-linking agent has been added to the suspension, heat may be applied to promote non-covalent bond cross linking between the cross-linking agent and the organic polymers. Specifically, 40-80°C, inclusive, of heat may be applied to the suspension for 0.5-2.5 hours, inclusive. During heating, silanol condensation among inorganic fillers may further stabilize the structure, when such fillers contain surface silanol groups. As a nonlimiting example, the cross-linked protective material may have a viscosity between 300-1.5x10⁴ mPa·s, inclusive. Upon reaching the final viscosity, the stabilized crosslinked protective material is created.

The cross-linked protective material may improve adhesion to plant surfaces. Accordingly, the cross-linking may improve the protective material durability, enabling it to last in varying environmental conditions, while simultaneously reducing the need for frequent reapplication. For instance, the cross-linking may allow the protective material to adhere to uneven plant surfaces and/or hydrophobic plant cuticles, unlike existing plant surface coatings.

Additionally, the final composition of the protective material may be as follows: (1) 0.1-2.5% w/w, inclusive, cellulose fibers (i.e., CMF/MFC); (2) 0.1-10% w/w, inclusive, silica microparticles; (3) 0.1-0.5% w/w, inclusive, silica particles having a diameter between 1-1000 nm, inclusive; (4) 0.18-0.22% w/w, inclusive, tannic acid; and (5) the remainder being comprised of deionized water. However, the final composition may also be comprised of trace amounts of the salt.

Each component of the protective material as described herein may play an important role in the overall functionality of the protective material. To illustrate, the organic polymers may provide structural support, flexibility, and/or transparency for light transmission, while also enhancing thermal radiation of the protective material as a whole. Moreover, the inorganic and/or ceramic fillers, embedded within the organic polymer matrix, may enhance the protective material emissivity by targeting specific IR wavelengths and optimizing cooling performance. Yet further, the porous structure may facilitate transient water retention, may improve mechanical stability, may support gas exchange, may contribute to light scattering, and may provide thermal insulation.

The protective material may also be comprised of adhesion promoters. For example, the adhesion promoters may be comprised of chemical groups and/or surface-active agents that improve the protective material adherence to plant surfaces. In a nonlimiting example, the adhesion promoters may include functionalized polymers or surfactants that form robust bonds with plant surfaces. Said adhesion promoters may ensure the protective material adheres to plant surfaces for long periods of time despite exposure to environmental conditions like rain or wind.

Non-limiting examples of adhesion promoters include tannic acid, organosilicone surfactants such as trisiloxane ethoxylates, as well as natural surfactants or adjuvants such as lecithin, saponins, and plant-derived oils or wax emulsions; and exopolysaccharides and biopolymers such as xanthan gum, dextran, gellan gum, alginate, kefiran, hyaluronic acid, bacterial cellulose, and levan.

The protective material described herein may be particularly well adapted for arid environments. To illustrate, the protective material is a water-independent solution for cooling plant surfaces. Meaning, vast quantities of water to cool plants is not required, as it would be for misting systems in arid climates.

As described above, the protective material ability to gradually release water enhances cooling via evaporation. In an embodiment, the protective material may prolong the period during which evaporative cooling occurs by maintaining transient moisture films, thereby enhancing the plant’s capacity to regulate its temperature.

Further, the protective material may attenuate portions of UV and IR radiation, which can help reduce photodamage and/or sunburn from occurring on plant surfaces due to prolonged sun exposure. The protective material ability to modulate both UV and IR radiation may contribute to improved long term health and productivity of plants in hot, humid, and sunny environments.

Moreover, the protective material may be used in an array of agricultural scenarios across a variety of crops and ornamentals. For example, the protective material may be used in open fields, controlled environments (e.g., greenhouses), and/or semi-controlled environments (e.g., shade houses). The protective material may be applied as a foliar spray directly and/or indirectly to plant surfaces, including leaves, stems, fruits and soil, mulch and/or structural elements in agricultural infrastructures. In preferred embodiments, the protective material is applied predominantly to the adaxial (upper) leaf surface, or in a partial or patterned coverage, so as to minimize interference with stomatal gas exchange. In such cases, the protective material forms a thin coating that adheres to the biological surface. This coating can provide surface level temperature modulation, radiative shielding, and protective barrier functionality. Such foliar sprayable deployment supports both preventative and responsive treatments and may be readily integrated into existing agricultural spraying practices.

Additionally, the protective material may be comprised of biocompatible and/or biodegradable materials. Said materials may be derived from organic waste. Further, the biocompatible and/or biodegradable materials may reduce the protective material impact on the environment by employing circular economy principles. Unlike traditional plant surface coatings, which require energy intensive processing to mine materials and manufacture the coating, the protective material described herein minimizes ecological harm by utilizing organic waste products to close a resource loop.

Further, the biocompatible and/or biodegradable materials comprising the protective material may have a degradation half-life of 60-90 days. In certain non-limiting embodiments, such degradation behavior may be observed under soil-burial conditions, for example at a soil moisture content of approximately 40% and a soil temperature of approximately 22°C. In such embodiments, the protective material may degrade into water, amorphous silica, and naturally occurring organic degradation products, such as oligosaccharides, organic acids, and monomers, which may ultimately mineralize to CO₂. In such a nonlimiting example, the protective material, while degrading, may remain compatible with microorganisms residing within the soil. Meaning, beneficial bacterial and fungal activity within the soil is not inhibited by the degradation of the protective material over time.

In yet a further embodiment, the protective material may form hydrogen bonds and/or Van der Waals interactions with the cuticle of a plant. In such an embodiment, said bonds and/or interactions may facilitate stable adhesion to plant surfaces, especially under stress from environmental conditions such as wind, rain, etc.

Yet further, under certain environmental conditions, the protective material ability to modify plant surface temperatures may contribute to reducing the risk of formation of frost. For example, by modulating optical and/or thermal properties, the protective material may act as a thermal insulation layer with low thermal conductivity and low emissivity. In embodiments containing optional phase-change domains, latent-heat buffering may occur, allowing stored heat to be released as temperatures fall and thereby moderating the cooling rate of the plant surface. In such embodiments, the organic polymer matrix of the protective material may interfere with ice nucleation by disrupting the formation of ice crystals on plant surfaces. Thus, the protective material maintains plant surface temperatures above the freezing point and/or dew point.

In further embodiments, the protective material may be utilized as a nutrient delivery system. To illustrate, the protective material may deliver micro and macro nutrients to plant surfaces, while maintaining its cooling functionality. In such an embodiment, controlled nutrient delivery may be facilitated by at least one of porosity modulation, ionic interactions, and osmotic diffusion. That is, the protective material may ensure efficient uptake of nutrients, while simultaneously minimizing the loss of nutrients via leaching. Moreover, encapsulated nutrients may be released via moisture-triggered swelling, pH-responsive ion exchange, and/or enzymatic hydrolysis, which optimizes bioavailability and/or root-zone retention.

Additionally, the protective material may be exploited as a detector and/or sensor material. To explain, the micro/nanocellulose-silica protective material may be functionalized with at least one of specific chemical probes and optical probes to serve as a sensor. Specifically, the protective material porous structure, and ability to host other molecules, may allow it to be exploited as a temperature sensor. For instance, the radiative cooling effect of the protective material may be tuned to detect and indicate temperature changes through optical and/or colorimetric shifts. Moreover, functionalizing the protective material with reactive molecules may allow said protective material to detect humidity, gases (e.g., CO₂, NH₃), and/or pollutants in either agricultural or environmental settings.

In further alternative embodiments, coating greenhouse panels in the protective material may reduce internal temperatures of the greenhouse while simultaneously maintaining light transmission for photosynthesis in plants.

The protective material may be applied to soil surfaces to form a functional mulch layer, or used to modify other surfaces, including films, panels, or structural elements in agricultural infrastructures, without requiring replacement or reconstruction of existing systems.

The protective material described herein may also be used for wound dressing and/or localized delivery of protective agents. For instance, the protective material cooling and protective properties may be adapted for plant wound care. In particular, the protective material may be used to address wounds requiring temperature control. Moreover, the micro/nanocellulose-silica matrix of the protective material may be leveraged for sustained release of therapeutic agents, especially in localized cooling treatments.

Similar to its application to greenhouses, the protective material may also be exploited for use in industrial food packaging. As a nonlimiting example, the protective material may be applied to food packaging, which necessarily cools said packaging, thus maintaining the freshness of temperature-sensitive goods.

Finally, other implementations of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

Various elements, which are described herein in the context of one or more embodiments, may be provided separately or in any suitable subcombination. Further, the processes described herein are not limited to the specific embodiments described. For example, the processes described herein are not limited to the specific processing order described herein and, rather, process blocks may be re-ordered, combined, removed, or performed in parallel or in serial, as necessary, to achieve the results set forth herein.

It will be further understood that various changes in the details, materials, and arrangements of the parts that have been described and illustrated herein may be made by those skilled in the art without departing from the scope of the following claims.

All references, patents and patent applications and publications that are cited or referred to in this application are incorporated in their entirety herein by reference. Finally, other implementations of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

### EXAMPLES

The following examples are put forth to provide those of ordinary skill in the art with a complete disclosure and description of how to make and use the invention of the present disclosure and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

### Example 1: Properties of an Exemplary protective material

### Composition of Exemplary protective material

A protective material was generated using the method described in FIG. 1. The tested samples covered formulations with a cellulose-based component on the order of ~0.1-1 w/w% and inorganic particulate fillers on the order of ~0.1–1 w/w% in the as-applied dispersion, together with optional low-level formulation aids (generally ≤0.5 w/w% each).

### Apparent viscosity characterization relevant to spray application

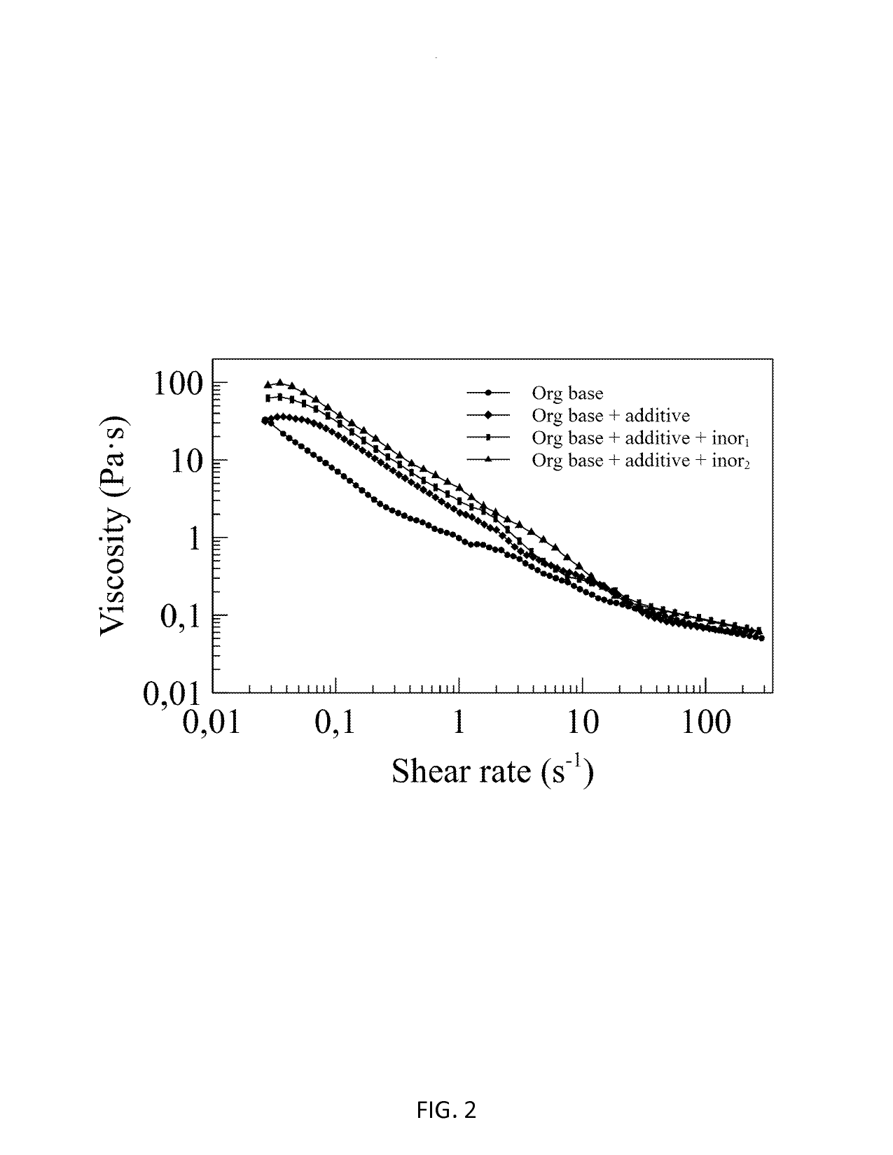

Apparent viscosity of example suspensions was evaluated as a function of shear rate to assess sprayability (FIG. 2). Samples comprising an organic polymer base dispersion were prepared with an optional formulation additive and different types of inorganic particulate fillers.

Apparent viscosity was measured using a rotational rheometer operated in controlled-shear mode, with shear rate swept over a range representative of conditions encountered during pumping, spraying, and atomization.

The example suspensions exhibited pronounced shear-thinning behavior, with apparent viscosity decreasing by more than an order of magnitude as shear rate increased. Such behavior is advantageous for spray application, as it enables reduced resistance to flow during pumping and atomization while maintaining higher viscosity under low-shear conditions. The data further shows that the inclusion of an optional formulation additive and/or different inorganic particulate fillers does not impair flowability, consistent with sprayability being maintained across different formulation compositions.

### Morphology of the protective material

The morphology of the protective material was examined by scanning electron microscopy (SEM). Dried coating were prepared for SEM imaging, and imaging conditions were selected to enable visualization of hierarchical microstructural features, including fibrous and particulate domains.

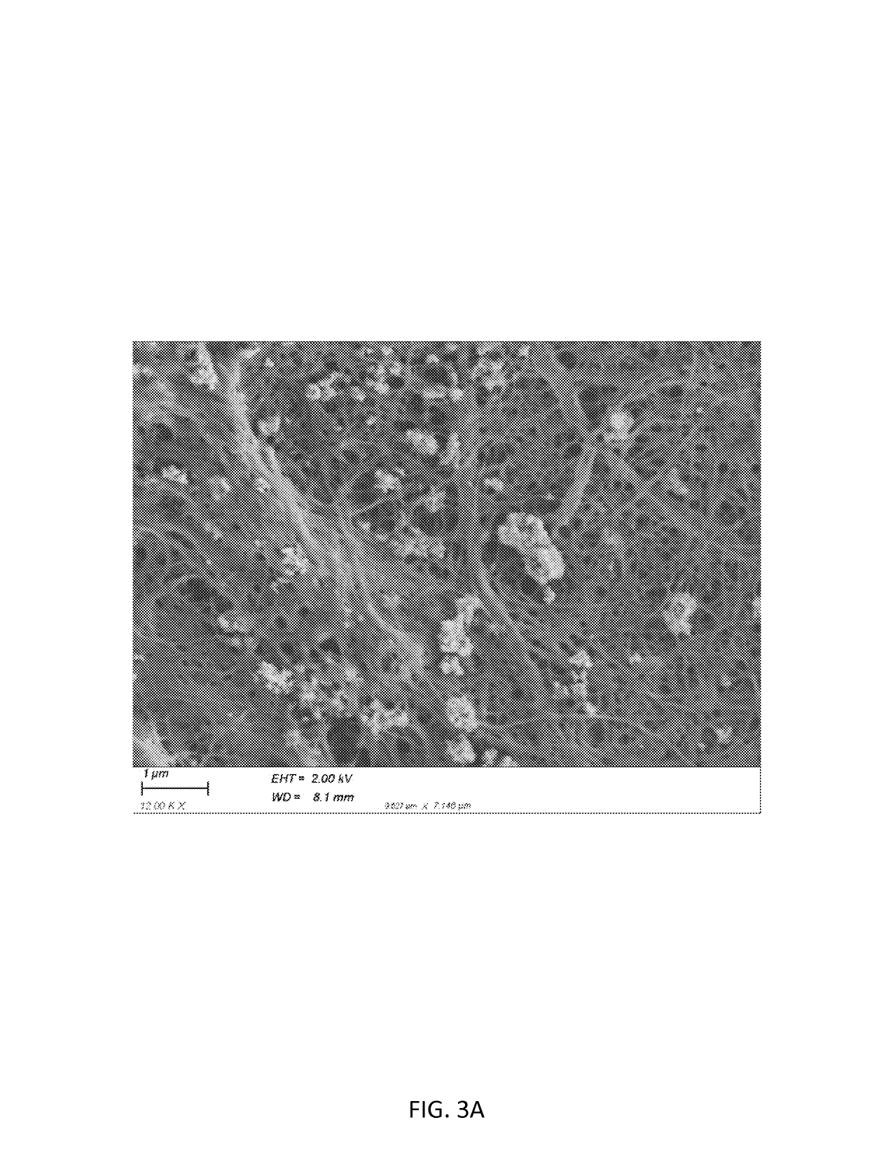

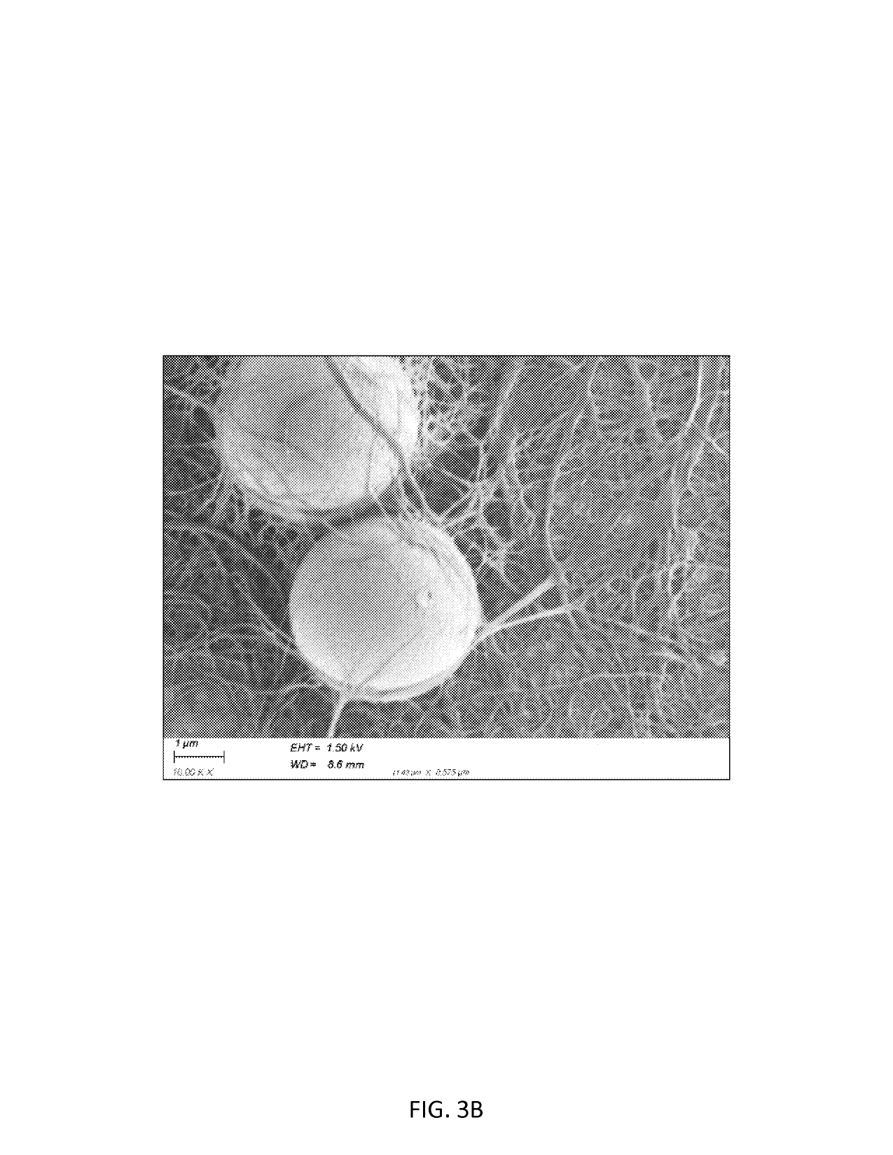

FIGS. 3A-3B illustrate exemplary microstructures of the protective material. FIG. 3A shows a porous fibrillar network with dispersed particulate aggregates, while FIG. 3B shows an alternative embodiment comprising bead-like particulate domains embedded within the fibrillar matrix.

The electron microscopy data indicates that representative dried coatings exhibit a porous, interconnected fibrillar network containing dispersed particulate domains, including alternative particulate morphologies across example embodiments.

The observed sub-micron to micron-scale void structure is consistent with a non-dense morphology that may support vapor and gas transport through the coating relative to continuous-film barriers.

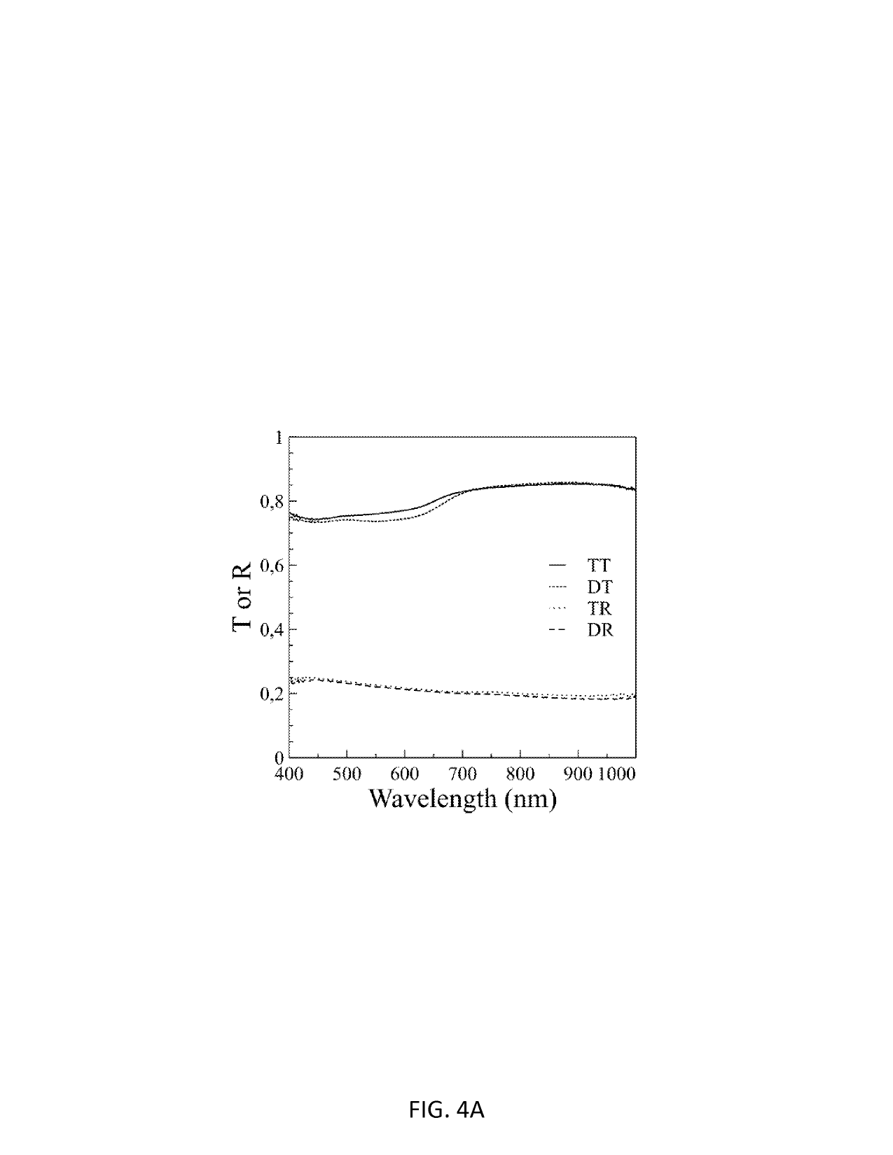

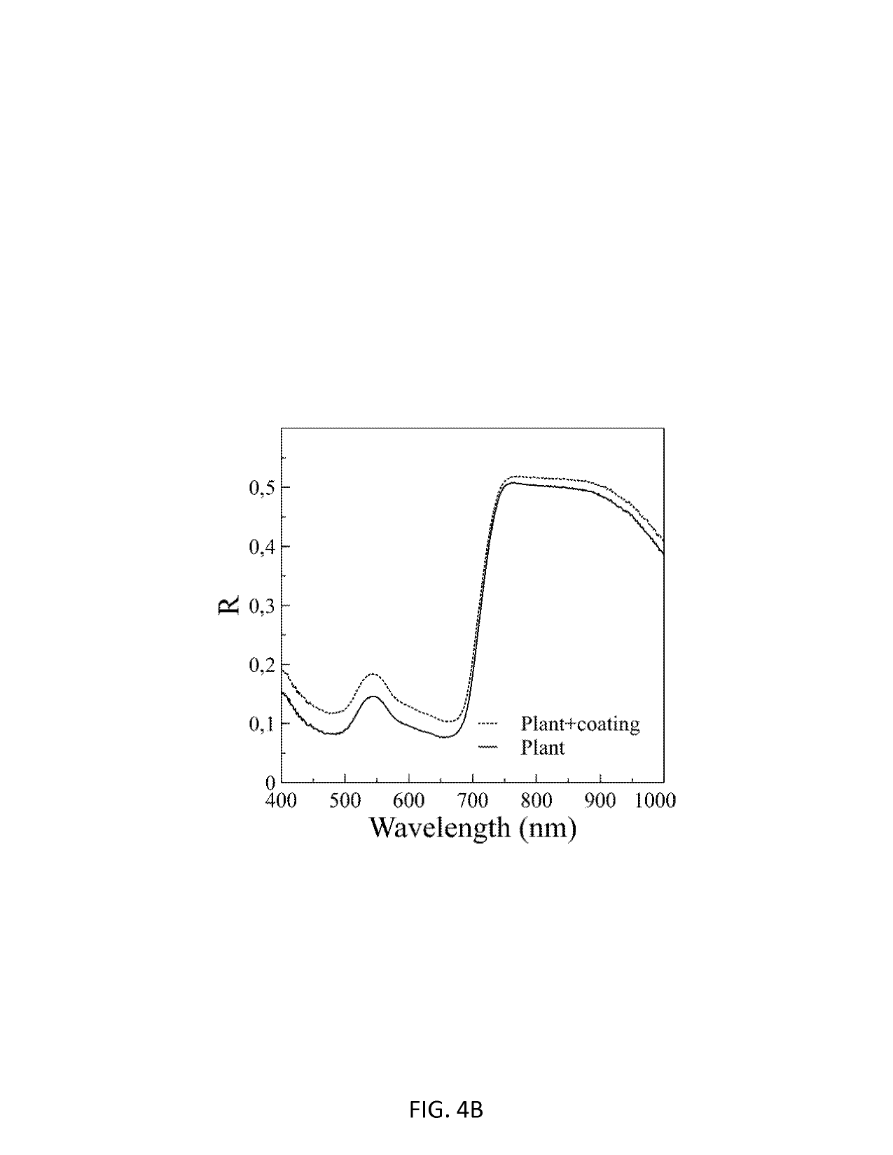

The multi-phase architecture (solid fibers/particles and voids) is consistent with enhanced optical scattering via refractive-index contrast, supported by optical measurements indicating predominantly diffuse transmitted light through representative coatings (FIG. 4A). In the dry- state, where voids are expected to be predominantly air-filled, the porous morphology is additionally consistent with reduced effective thermal conductivity relative to a dense coating of similar composition and thickness due to interruption of continuous solid heat-transfer pathways by low-conductivity air gaps.

The microscopy data supports a coating design intended to provide open-porosity pathways, diffuse optical scattering, and potentially reduced dry-state conductive heat transfer features, that can contribute to heat-stress mitigation when applied to plant surfaces.

### Transmittance and Reflectivity of the Protective material

Optical properties of the protective material were measured over the Vis-NIR range (400-1000 nm). Coatings were deposited as uniform films on optically transparent substrates and analyzed using an integrating sphere to obtain total and diffuse components of transmittance and reflectance. Leaf-level total reflectance measurements were performed on fresh coffee leaves, with spectra acquired immediately after excision. For each leaf, the coating was applied to one half of the same leaf while the other half was left uncoated and used as a control.

FIGS. 4A-4B show optical spectra showing total and diffuse transmittance and reflectance components (TT, DT, TR, DR) of a protective material measured over 400-1000 nm (FIG. 4A) and total reflectance of fresh coffee leaves with and without the coating (FIG. 4B).

Optical characterization reveals that the exemplary protective material can have a favorable balance of light-management properties relevant to both photosynthetic performance and thermal regulation. The film exhibited high total transmittance across the measured range, with the transmitted component being predominantly diffused, a feature that may redistribute PAR more uniformly and may reduce localized irradiance and photoinhibition.

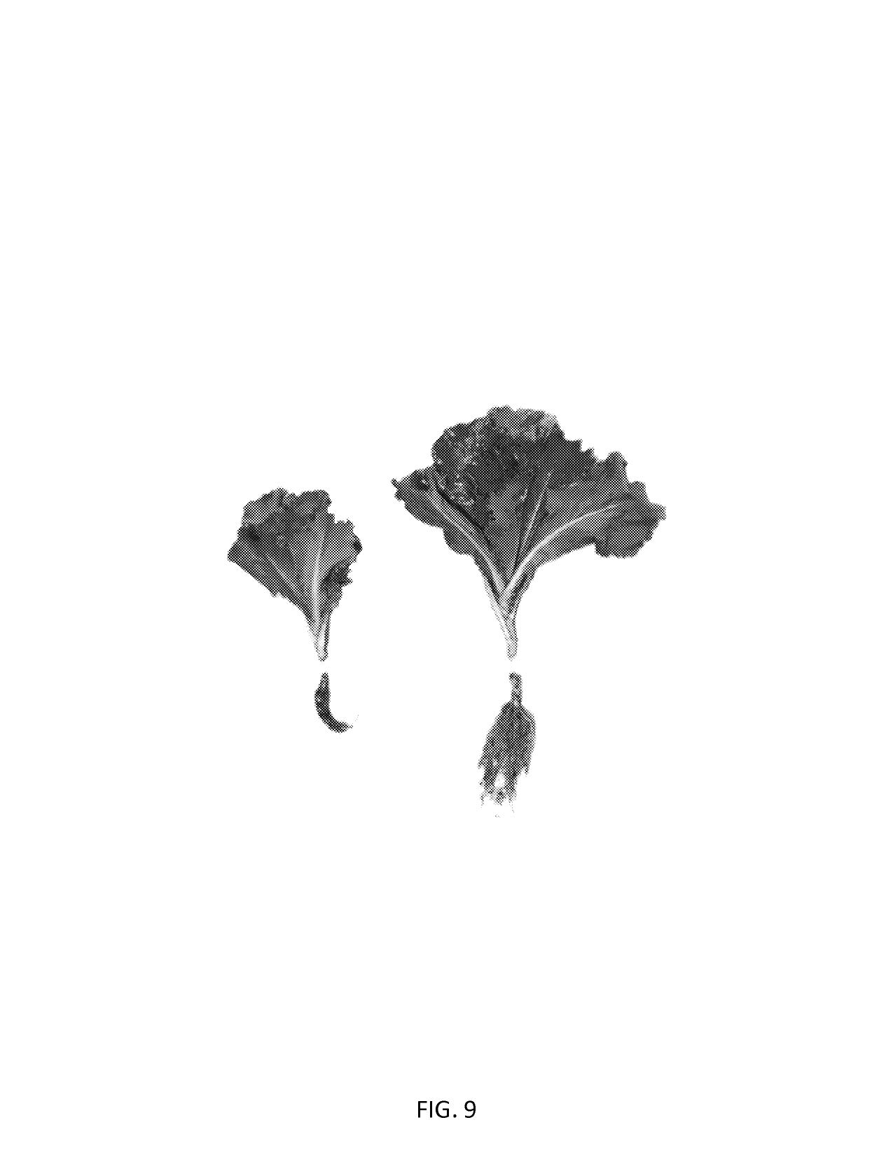

In this sense, the protective material may act as a modulating optical layer, altering the angular and spectral distribution of incident radiation rather than acting as a light-blocking screen. Diffuse transmission of PAR has been reported in the literature to improve intra-canopy light distribution and, in certain cropping systems, to support increased canopy photosynthesis and/or growth (Li & Yang, 2015); consistent with this framework, the exemplary protective material described herein exhibited predominantly diffuse transmission, and corresponding plant-growth responses are further described in FIG. 9.

Representative protective material further exhibited measurable total reflectance (e.g., in the order of about 20-30% in certain embodiments within at least a portion of the measured range), with a substantial diffuse component that may contribute to partial rejection of non-photosynthetically useful radiation while avoiding specular reflection that could lead to localized thermal hotspots.

In example embodiments, the combination of (i) predominantly diffuse transmission and (ii) diffuse reflectance is consistent with a microstructure comprising multiple phases and voids, as observed in electron microscopy images of representative dried coatings (FIGS. 3A-3B).

In measurements of fresh coffee leaves, the coated region exhibited slightly higher total reflectance relative to an uncoated control across portions of the visible and near-infrared wavelength range, while the overall spectral profile remained vegetation-like in the measured range. The observed reflectance behavior may be attributable, at least in part, to coating-induced surface and/or near-surface scattering effects, rather than a non-obvious pigment-signature disruption.

In general, increases in leaf reflectance have been reported under certain environmental conditions, including water limitation and/or elevated temperature. Such reflectance changes have been associated in the literature with endogenous protective responses and/or morphological or surface-property changes, for example, changes in cuticle thickness, epicuticular wax deposition, and/or increased pubescence, which can increase reflectance and reduce absorbed radiative loading (Camarillo-Castillo, et al., 2021).

In this context, in certain embodiments, the disclosed coating may provide an optical effect that is similar to one or more of these reported protective surface effects by modulating leaf reflectance and scattering, thereby providing partial modulation of leaf optical properties without requiring activation of stress-associated morphological adaptations.

### Emissivity of the Protective material

Infrared optical properties of example coatings prepared from different protective material formulations were measured in the mid-IR spectral range. Coatings were deposited on opaque substrates, including aluminum or silicon wafers, such that transmittance in the measured wavelength range was negligible. Total reflectance spectra were obtained using a Varian 3100 FT-IR - Excalibur Series spectrometer equipped with a liquid-nitrogen-cooled MCT detector and a PIKE Upward IntegratIR™ ATR accessory. Spectral emissivity was calculated from the measured reflectance based on Kirchhoff’s law of thermal radiation. For plant surface measurements, fresh leaves were excised immediately prior to analysis and measured without drying or storage. For each leaf, the coating was applied to one half of the same leaf while the other half was left uncoated and used as a control.

An effective foliar coating for thermal management must satisfy multiple, and sometimes competing, optical requirements. In addition to transmitting PAR while ideally selectively attenuating UV and portions of the NIR spectrum, the coating should preserve the intrinsic thermal emissivity of plant surfaces. Because native leaf emissivity in the mid-IR is already high, the principal functional requirement for a foliar coating is avoiding reduction of emissivity within the atmospheric transparency window, particularly by preventing the introduction of spectral penalties in specific mid-IR sub-bands.

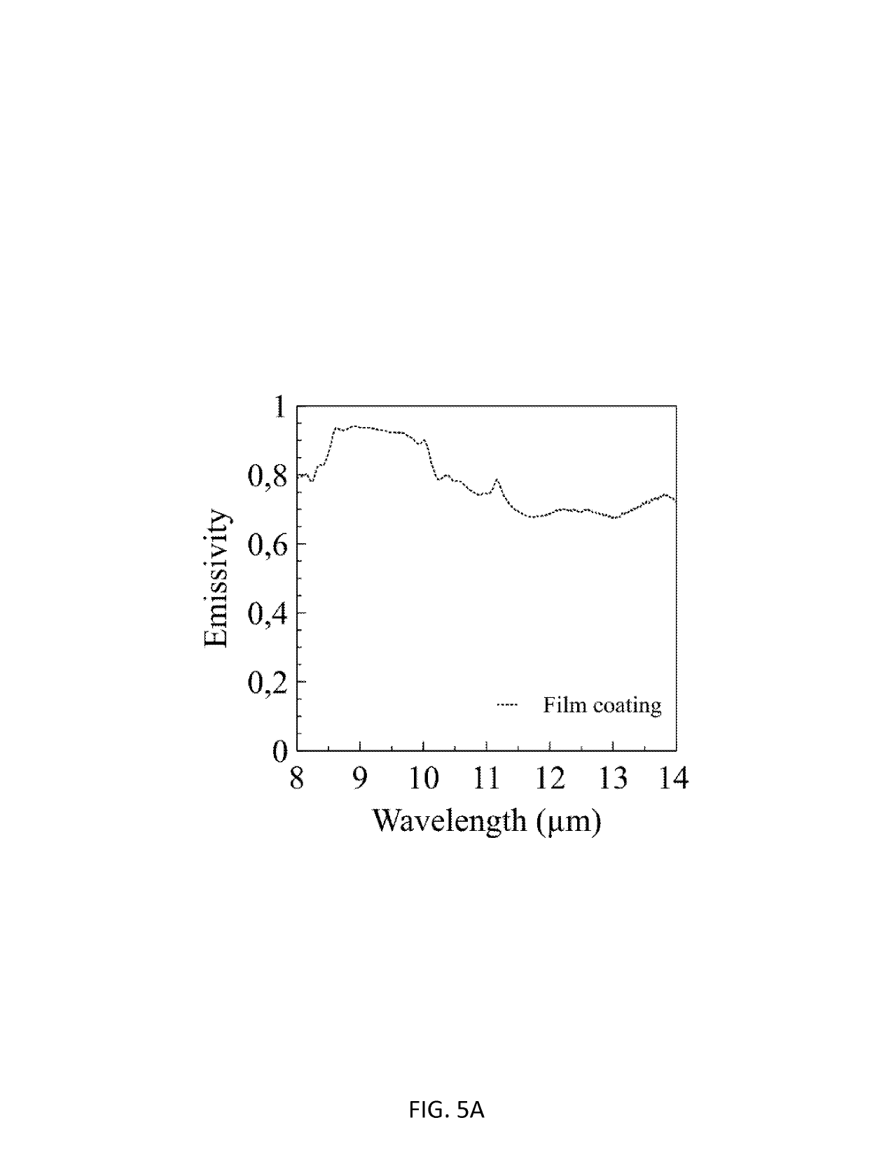

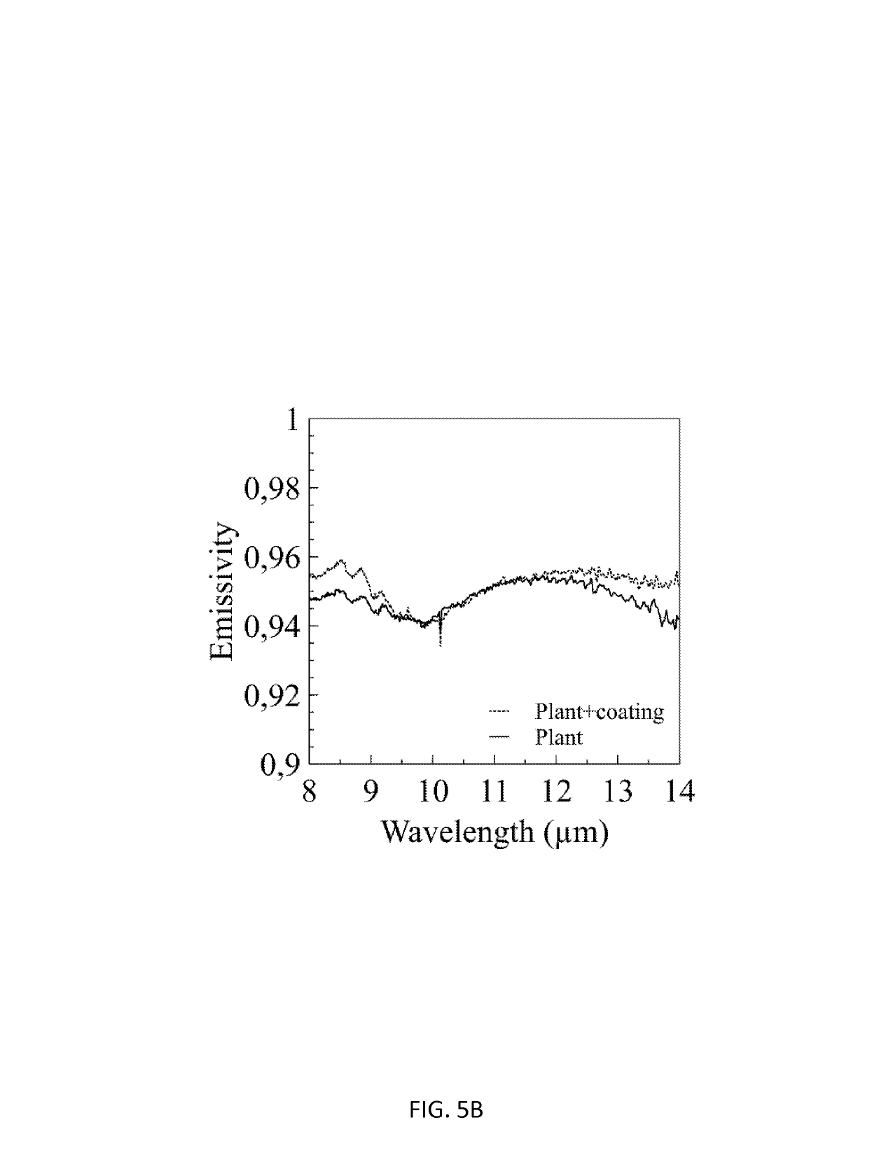

FIGS. 5A-5B show spectral emissivity in the mid-IR atmospheric transparency window (8-13 µm) for a dried coating formed from an example formulation (FIG. 5A), and an uncoated tomato leaf surface and a coated tomato leaf surface with an exemplary formulation coating (FIG. 5B). Note: where multiple formulations were evaluated, spectra may be presented for a representative formulation as an example, while measurements for additional formulations were obtained using the same protocol and instrumentation.

The representative dried coating exhibited elevated mid-IR spectral emissivity across portions of the 8-13 µm atmospheric transparency window relevant to radiative cooling. In the example embodiment, emissivity remained elevated (≥0.8) over one or more sub-ranges within the window, and in some sub-ranges approached or exceeded 0.9, supporting efficient radiative heat exchange with the sky.

FIG. 5B shows that the uncoated leaf surface already exhibited high and relatively flat mid-IR emissivity, consistent with known thermal-emissive nature of many biological surfaces. When applied to plant surfaces, the material coating can preserve the inherently high mid-IR emissivity of the leaf surface and, in certain wavelength sub-ranges, can locally enhance effective emissivity relative to the uncoated leaf, thereby maintaining compatibility with radiative heat dissipation from plant surfaces.

### Temperature Reduction of the Protective material

### Field-Relevant Demonstration

Heat-stress mitigation performance of a representative coating formulation was evaluated by time-resolved leaf surface temperature measurements under outdoor conditions. The coating was applied to one half of the same upper-canopy leaf (coating thickness 68 ± 9 µm), while the other half was left uncoated and used as a control. Adaxial leaf temperatures were monitored using contact leaf sensors (LAT-B3 sensors connected to DL 18 HOBO data logger) positioned to provide comparable placement and orientation for the coated and uncoated sections. Measurements were collected at one-minute intervals over 90 minutes. Local air temperature was recorded immediately above the leaf surface (3.5 cm above the leaf) and used as a boundary-layer reference. Measurements were conducted on an outdoor fig tree under clear sky conditions with no enclosure or shade, such that leaves were fully exposed to direct solar radiation, wind-driven convection, and ambient humidity.

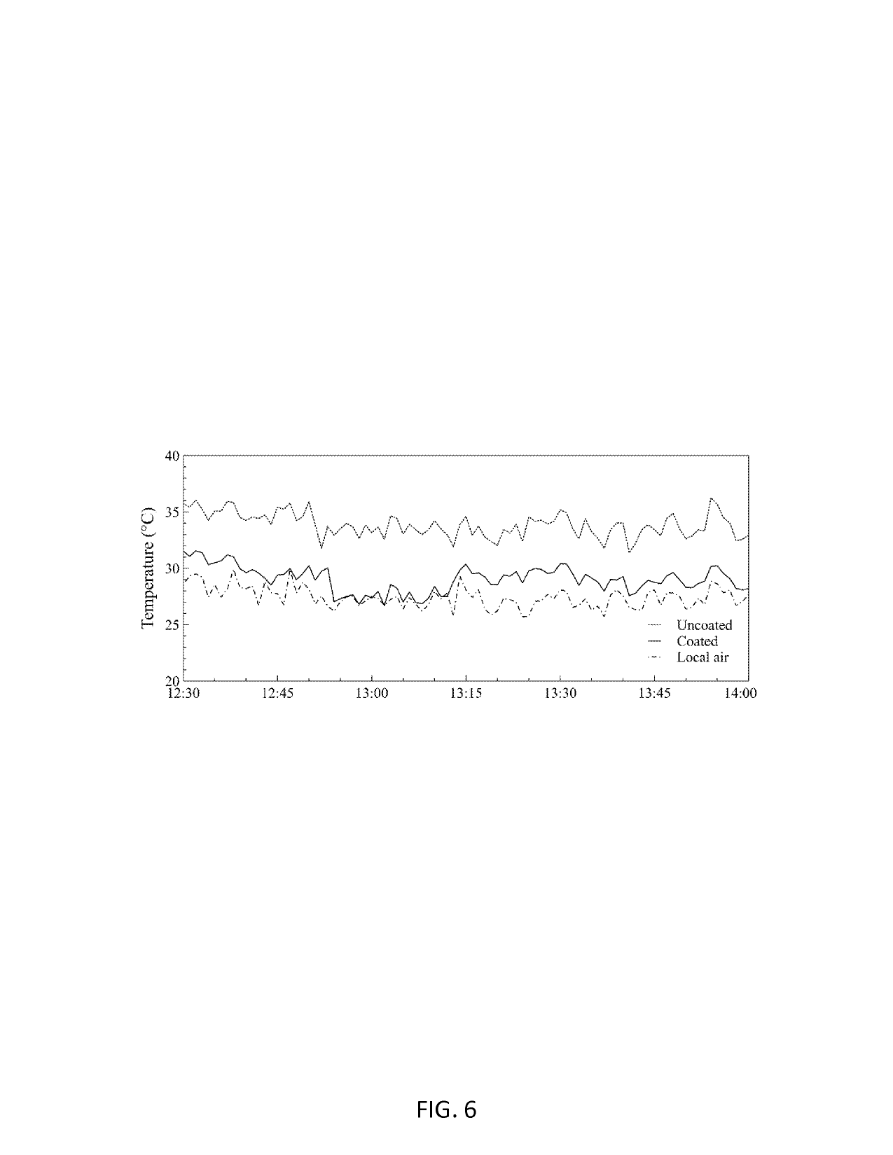

FIG. 6 shows measured leaf temperature as a function of time for half-uncoated and half-coated regions of the same upper-canopy fig tree leaf under outdoor exposure.

Under open‑sky, fully exposed outdoor conditions, with incident solar irradiance between 890-900 W·m⁻², relative humidity of ~68%, and wind speed up to 2.3 km·h⁻¹ (all measured at the test site), the coated section of the leaf exhibited a lower measured surface temperature than the uncoated leaf section over the recorded interval. The observed temperature reduction under matched exposure is consistent with a coating‑associated change in leaf surface energy balance that increases net heat dissipation and/or reduces net heat gain under the tested conditions. Given that wind‑driven convection, transpiration, and time‑varying irradiance can contribute under fully exposed outdoor conditions, this result is most appropriately interpreted as a field‑relevant performance demonstration rather than as attribution to a single cooling mechanism.

### Controlled/Mechanistic-Supporting Demonstration

Temperature measurements were conducted to obtain a mechanistic-supporting comparison of coated and uncoated leaf surface temperatures under outdoor irradiance while reducing variability arising from wind-driven convection. The test configuration employed a rigid board structure configured to reduce wind-driven convection while maintaining radiative coupling to the sky. The enclosure exterior was wrapped with reflective Mylar to limit parasitic heating of the structure. The top opening was covered with a polyethylene film intended to suppress convection. Side louvers were incorporated to allow air exchange while shading lateral solar inputs (Yang et al., 2020). The plants remained inside the board without sealing (pseudo-stagnation), thereby limiting formation of a confined humid air pocket. Soil moisture was maintained at same levels to reduce differences in root zone heat flux. A coated leaf (coating thickness 73 ± 12 µm) and an uncoated control leaf of similar developmental stage and canopy position (upper canopy) were selected from coffee seedlings. Adaxial leaf temperatures were monitored using contact leaf sensors (LAT-B3 sensors connected to DL 18 HOBO data logger) positioned to provide comparable placement and orientation for the coated and uncoated leaves. Measurements were collected at one-minute intervals over 90 minutes. Local air temperature was recorded immediately above the leaf surface (3.5 cm above the leaf) and used as a boundary-layer reference.

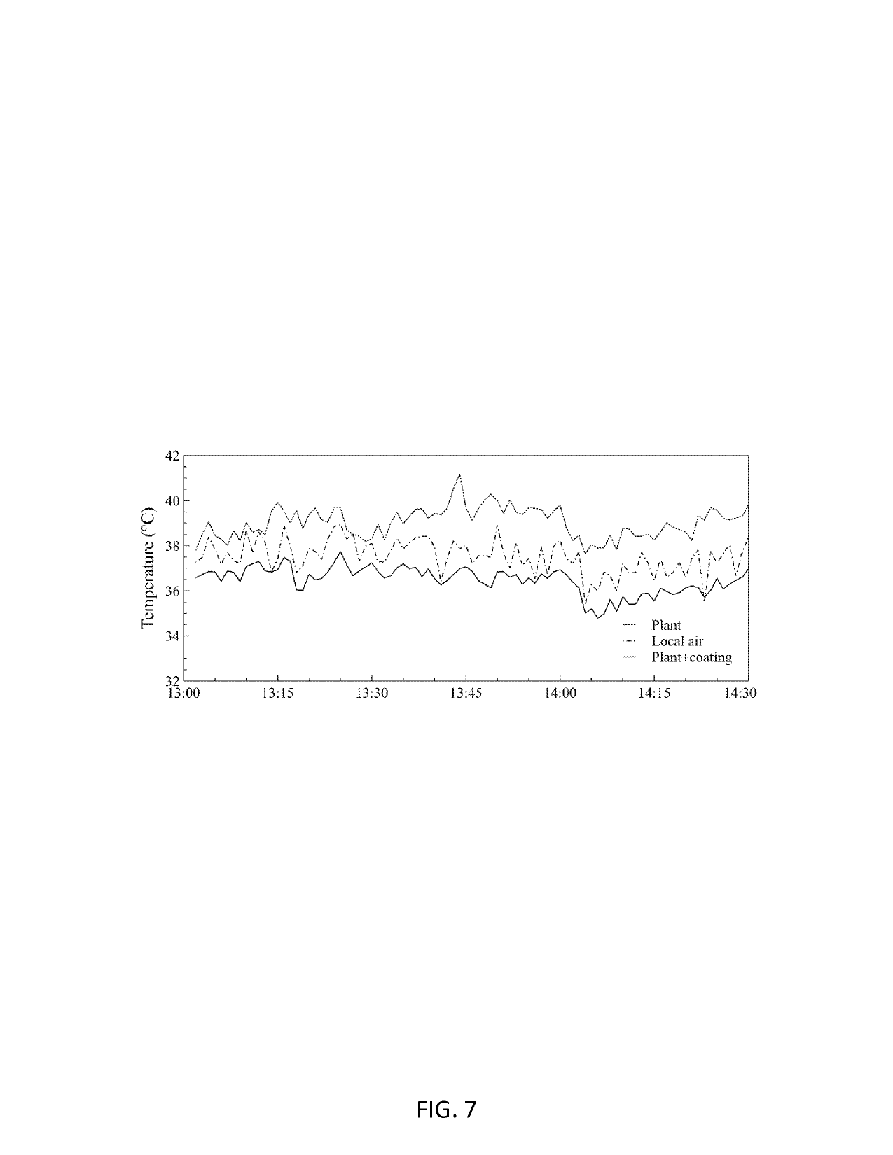

FIG. 7 shows measured leaf temperature as a function of time for an uncoated coffee plant and a coated coffee plant during exposure in a ventilated pseudo-stagnation enclosure.

In the convection‑mitigated, sky‑coupled enclosure test, with incident solar irradiance between 960-970 W·m⁻², relative humidity of ~50%, and wind speed up to 25 km·h⁻¹ (all measured at the test site), the coated leaf exhibited a lower measured surface temperature than the uncoated control leaf over the measurement period. For at least portions of the interval, the coated‑leaf temperature was at or below the local air temperature measured proximate to the leaf surface. In a configuration designed to reduce wind‑driven convective exchange while maintaining radiative coupling to the sky, such sub‑air temperature intervals indicate net heat loss from the coated leaf surface and are consistent with increased radiative heat loss to the sky and/or other coating‑enabled heat‑dissipation pathways, without excluding contributions from transpiration and/or reduced solar heat gain due to coating‑modified reflectance and/or scattering.

### Thermal Imaging

Potted cherry tomato plants of the same variety and comparable developmental stage were evaluated under matched outdoor conditions: (i) control (uncoated plant) and (ii) treated (plant coated with a representative formulation). Both plants were maintained under the same outdoor exposure and watering history prior to measurement. Plants were positioned in the same local area to minimize spatial gradients in irradiance and ambient conditions. Apparent leaf surface temperature was measured using an infrared thermal imaging camera operating in the long-wave infrared range (TOPDON TC004).

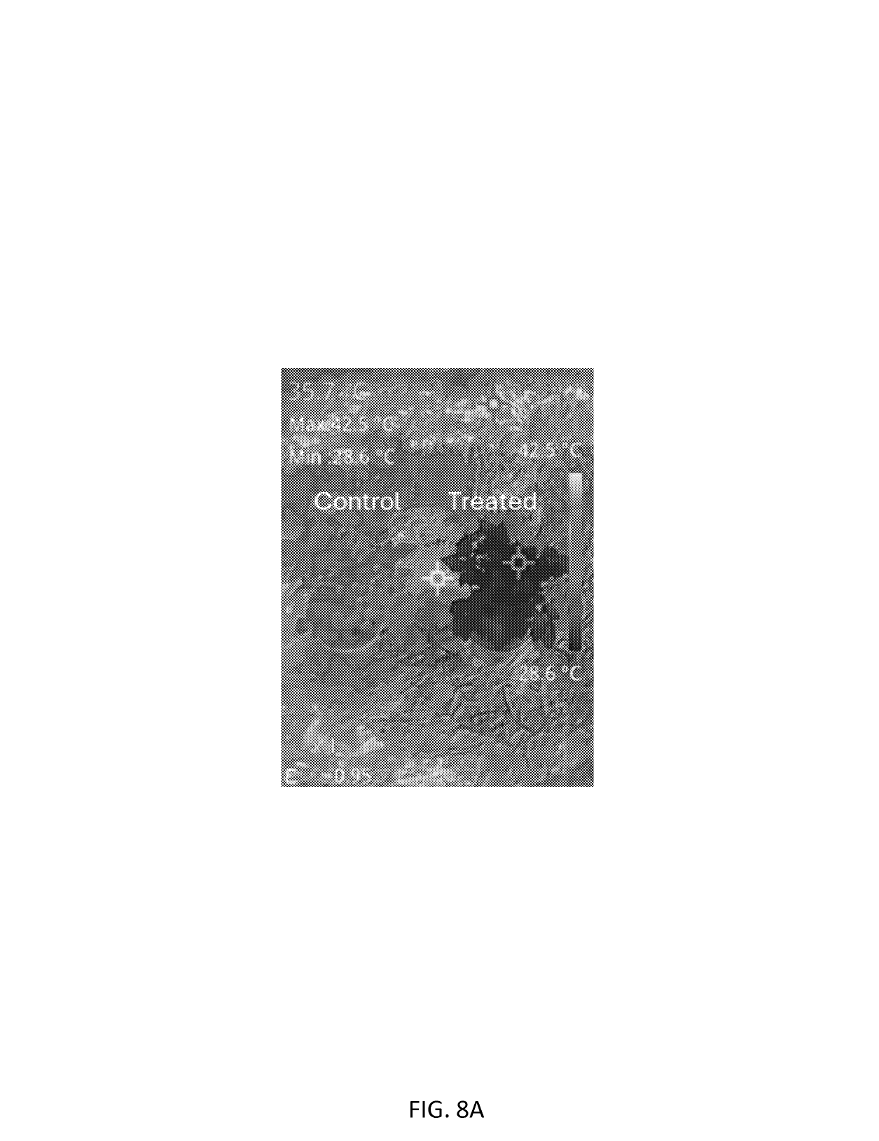

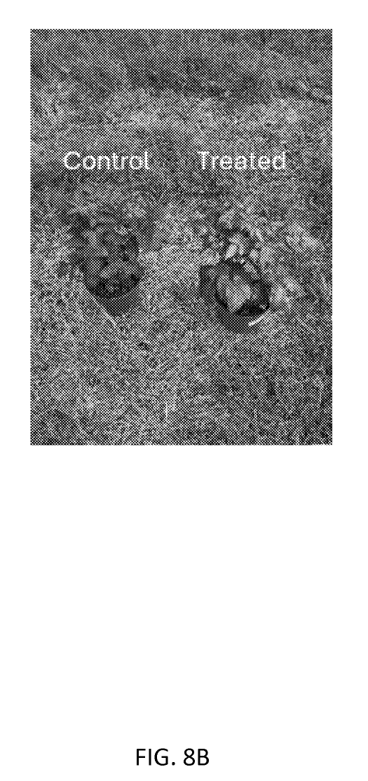

FIGS. 8A-8B illustrate infrared thermography (FIG. 8A) and corresponding photograph (FIG. 8B) of potted cherry tomato plants under outdoor irradiance (862 W·m⁻²), ambient air temperature 33 °C, relative humidity 66%, and equal soil humidity (85%). The control (uncoated) plant is shown on the left and the treated (coated) plant is shown on the right. IR images captured at 1 m distance and emissivity setting ε = 0.95.

Leaf temperature was quantified by thermal spot measurements collected from fifteen (n = 15) matched regions on the upper adaxial leaf surfaces of each plant. Spot locations were selected to be comparable with respect to canopy position and sun exposure and were placed on leaf lamina.

Under the stated outdoor irradiance and environmental conditions, infrared thermography indicated that the treated (coated) plant exhibited a lower apparent leaf surface temperature than the matched control (uncoated) plant, consistent with a temperature reduction of ΔT = 4.8 ± 0.7 °C.

### Plant Growth

Lettuce plants were cultivated under controlled, non-commercial conditions without fertilization or intensive agronomic management to isolate the effect of a treatment comprising an embodiment of the disclosed protective material. Treated plants and untreated control plants were grown under identical environmental conditions, with n = 9 plants per group. At approximately eight weeks after sowing, shoots were harvested at the crown and roots were uprooted, rinsed, patted dry, and fresh biomass was measured separately for shoots and roots.

Under the tested conditions, plants treated with the protective material exhibited increased fresh biomass relative to untreated controls. Shoot fresh mass increased by 40% (p < 0.01) and root fresh mass increased by 53% (p < 0.05) compared to the control group.

FIG. 9 illustrates representative images of lettuce plants grown under controlled conditions, including an untreated control plant (left) and a plant treated with an embodiment of the protective material described herein (right). Shoots and roots are shown after harvest for visual comparison of plant morphology and fresh biomass.

The statistically significant increases in shoot and root fresh mass are consistent with compatibility of the treatment with plant growth and, under the tested conditions, do not indicate gross phytotoxicity or growth inhibition. The observation that both shoot and root biomass increased is also consistent with an absence of a strong growth trade-off that might otherwise suggest stress-associated biomass reallocation.

In non-limiting examples, absence of growth inhibition and/or absence of readily observable visible damage (e.g., severe leaf necrosis, gross deformation, or marked growth suppression) following treatment may be used as an indication that a formulation is compatible with plant surfaces under the tested conditions.

### References

Camarillo-Castillo, F., Huggins, T. D., Mondal, S., Reynolds, M. P., Tilley, M., & Hays, D. B. (2021). High-resolution spectral information enables phenotyping of leaf epicuticular wax in wheat. Plant Methods, 17(1).

Li, T., & Yang, Q. (2015). Advantages of diffuse light for horticultural production and perspectives for further research. In Frontiers in Plant Science (Vol. 6, Issue september). Frontiers Research Foundation.

Yang, Y., Long, L., Meng, S., Denisuk, N., Chen, G., Wang, L., & Zhu, Y. (2020). Bulk material based selective infrared emitter for sub-ambient daytime radiative cooling. Solar Energy Materials and Solar Cells, 211.

What is claimed is:

1. A protective material for heat stress mitigation in plants, wherein the protective material comprises a hybrid composite comprising:
(i) a plurality of organic polymer-based dispersions dispersed in a treated liquid;
(ii) an inorganic and/or ceramic filler mixed with the plurality of organic polymer based dispersion,
wherein the inorganic and/or ceramic filler comprises a plurality of microparticles, a plurality of submicron particles, a plurality of nanoparticles, or a combination thereof; and
wherein the protective material is configured, upon drying, to form a hybrid composite coating having an organic matrix with embedded inorganic and/or ceramic fillers, the coating being adapted to mitigate heat stress on plant surfaces by reducing solar heat gain, support radiative cooling, and managing heat transfer.

2. The protective material of claim 1, wherein the protective material is provided as a suspension in the treated liquid, wherein the suspension exhibits shear-rate-dependent apparent viscosity selected to enhance sprayability.

3. The protective material of claim 1, wherein the inorganic and/or ceramic filler is operable to support vibrational or phonon-polariton-enhanced thermal emission within the mid-infrared region.

4. The protective material of claim 1, wherein each microparticle of the plurality of microparticles has a diameter between 1-100 µm.

5. The protective material of claim 1, wherein each submicron particle of the plurality of submicron particles has a diameter between 100-1000 nm.

6. The protective material of claim 1, wherein each nanoparticle of the plurality of nanoparticles has a diameter between 1-100 nm.

7. The protective material of claim 1, wherein, when dried on a substrate, the protective material forms a radiative functional coating configured to facilitate thermal-infrared radiative heat transfer within an atmospheric transparency window.

8. The protective material of claim 1, wherein the coating is configured to selectively modulate incident solar irradiance by reflecting or scattering radiation.

9. The protective material of claim 8, wherein the solar irradiance comprises transmitted photosynthetically active radiation (PAR), and wherein the PAR is predominantly diffuse.

10. The protective material of claim 1, wherein the coating has a thickness from about 1 µm to about 100 µm.

11. The protective material of claim 1, wherein the organic polymer-based dispersion comprises at least one of cellulose microfibers, microfibrillated cellulose, cellulose nanofibers, microcrystalline cellulose, carboxymethyl cellulose, and biodegradable synthetic polymers,
wherein the biodegradable synthetic polymers comprise aliphatic polyesters and water-soluble synthetic polymers comprising poly(lactic acid) (PLA), polyhydroxyalkanoates (PHA), and poly(vinyl alcohol) (PVA).

12. The protective material of claim 11, further comprising polysaccharide- or biopolymer-based thickening agents, chitosan, alginate, pectin, pullulan, lanolin, xanthan gum, natural waxes, carnauba wax, stearic acid, beeswax, natural paraffins, candelilla wax, or combinations thereof.

13. The protective material of claim 1, wherein the inorganic and/or ceramic filler comprises titanium dioxide (TiO₂), silica (SiO₂), calcium oxalate (CaC₂O₄) , zinc oxide (ZnO), iron oxide (Fe₂O₃), aluminum oxide (Al₂O₃), magnesium oxide (MgO), zeolitic materials, or a combination thereof.

14. The protective material of claim 1, wherein:
(a) the organic polymer-based dispersion comprises 0.1-2.5% w/w cellulose fibers; and
(b) the inorganic and/or ceramic filler comprises 0.1-10% w/w silica microparticles.

15. A method of manufacturing a protective material for mitigating heat stress in plants, the method comprising the steps of:
(a) preparing an organic polymer base dispersion; and
(b) introducing to the organic polymer base dispersion, at least one quantity of an inorganic and/or ceramic filler suspension, thereby forming a suspension.

16. The method of claim 15, further comprising inducing pore formation and structural development within the suspension.

17. The method of claim 15, further comprising cross-linking the suspension to enhance stability, thereby forming a crosslinked hybrid composite.
